(19) United States
(12) Patent Application Publication
(10) Pub. No.: US 2005/0059023 A1
(43) Pub. Date: Mar. 17, 2005

(54) Methods and kits for monitoring resistance to therapeutic agents

(76) Inventor: Thomas L. Cantor, El Cajon, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/664,263

(22) Filed: Sep. 16, 2003

(51) Int. Cl. C12Q 1/68; G01N 33/53

(52) U.S. Cl. 435/6; 435/7.1

(57) ABSTRACT

The present invention relates to novel methods and kits for monitoring the therapeutic inactivating capacity of a subject. Moreover, the present invention further relates to methods and kits for determining and/or monitoring a therapeutic protocol for a subject afflicted with auto antibodies specific for a natural substance, wherein these auto antibodies develop as a result of therapeutic administration of the natural substance or an analog thereof. These methods and kits can be used, for example, to initiate, terminate, or adjust the level of administration of any of a variety of therapeutic agents.

### I. TECHNICAL FIELD

The present invention relates to novel methods and kits for monitoring the therapeutic inactivating capacity of a subject. These methods and kits can be used, for example, to initiate, terminate, or adjust the level of administration of said therapeutic agent.

### II. BACKGROUND OF THE INVENTION

Any of a variety of therapeutic agents have the potential to trigger an adverse immunological response thereto in a subject; and traditionally, protein therapeutics run the highest risk of inducing such responses.

As one known example, monoclonal antibodies are often utilized in the treatment of disease. The source of the antibodies is important to determine whether non-self type immunological reactions will develop in response to their therapeutic administration. One such documented type of response in selected subjects is a reaction to mouse derived monoclonal antibodies characterized as a human anti-mouse immunological response, or the development of human anti-mouse antibodies (HAMAs). In this type of response, when mouse monoclonal antibodies are administered to a human subject, they are recognized as non-human and, over time, antibodies specific for the mouse antibodies are developed. Methods have been developed for detection of HAMAs. For example, Scantibodies Laboratory, Inc (Santee, Calif.) commercializes an immunoassay kit to quantitate the level of HAMA in a patient. Although the development and use of humanized antibodies has helped avoid the development of HAMAs, such humanized antibodies may also be recognized as foreign and result in human anti-human antibodies specific for the therapeutic humanized antibodies. Recently, methods have been discussed for the detection of an adverse immunological response, consisting of human anti-human antibodies generated as a result of the prior administration of humanized monoclonal antibody A33, utilizing surface plasmon resonance. See U.S. 2003/0040027 A1.

Exogenous administration of therapeutic hormones also have the potential to generate a non-self immunological response. Monitoring of such responses is important to ensure efficacy of the hormones and to avoid or monitor and control adverse immunological reactions. One example of this type of reaction involves the development of antibodies to exogenously administered insulin in diabetic patients. Determining whether auto antibodies are present specific for insulin could alter the therapy, i.e., by utilizing another therapeutic in lieu of insulin, e.g., GLUCOPHAGE® (Merck Santé S. A. S., an associate of Merck KGaA of Darmstadt, Germany). Further examples of recombinant hormones that are utilized as therapeutics include: GONAL® (human follicle stimulating hormone) (Serono S. A., Geneva, Switzerland), OVIDREL® (chorionic gonadotropin alfa) (Serono S. A.) and OVITRELLE® (human chorionic gonadotropin) (Serono S. A.), LUVERIS® (luteinizing hormone), CETROTIDE® (gonadotropin hormone releasing hormone) (Serono S. A.), all of which have the potential to elicit an immune response from the patient that could inactivate the therapy and set the patient at risk of an anaphylaxis reaction.

Leading to the present disclosure, it was recognized that some pathologies involve the over production of hormones, e.g., in renal failure, parathyroid hormone (PTH) is overproduced. In certain of these subjects, the body attempts to adapt to the increased production of hormone and develops auto antibodies against the hormone. Thus, the present disclosure provides methods and kits useful for identifying situations where autoimmunity is developed against specific hormones which is valuable to take into account for altering interventional therapy accordingly.

The present disclosure also recognizes that non-self immunological reactions can develop against chemical moieties utilized as therapeutics. Most, if not all, exogenously administered chemical therapeutics are foreign to a subject's body. As such, neutralizing antibodies can be developed against these therapeutics. In leading to the present disclosure, the inventor has recognized the therapeutic advantages of determining whether such reactions, or the potential for such reactions are present in a subject and altering therapy accordingly.

The utilization of naturally occurring substances as therapeutics can induce the production of neutralizing antibodies in a subject. Examples such as administration of erythropoietin and insulin as well as hormone replacement therapy often can lead to the development of neutralizing antibodies. Most such naturally occurring substances can be synthetically produced and the manner of production of these substances can have a large impact on the resulting immunological response in a subject. In addition, the presence of contaminants in naturally occurring substance therapeutics (such as fetal bovine serum, cell media, etc.) can often act as adjuvants for the production of neutralizing antibodies to naturally occurring substances. Nevertheless, it has been indicated that recombinant EPO itself may not be the primary cause of the development of anti-EPO antibodies; rather, modes of administration and/or by-products resulting from storage or formulation may be to blame. See Kai-Uwe Eckardt, et al., Nephrol. Dial. Transplant 18:865-69 (2003); Robert S. Hillman, Hematopoietic Agents—Growth Factors, Minerals, and Vitamins, in THE PHARMACOLOGICAL BASIS OF THERAPEUTICS 1312-15 (J G Hardman, et al. eds., 9th ed. 1996); product inserts for EPOGEN® (Epoetin alfa) (available on the internet at www.renaladvances.com/PDF/EpogenPI.pdf), PROCRIT® (Epoetin alfa) (available on the internet at healthcareprofessionals.orthobiotech.com/products/procrit/procrit.pdf), and EPREX® (epoetin alfa, Ortho Biologics, LLC); and Johnson & Johnson Press Release “Summary of PRCA Case Reports,” Jul. 14, 2003 (available at wwwjnj.com/news/jnj_news/1021024_{—}095632.htm).

Auto antibodies can also develop in a subject that are specific for cell surface receptors. Such auto antibodies can often mimic or block the actions of ligand that is specific for a given receptor. For example, auto antibodies against a calcium sensing receptor have been shown to mimic hypocalciuric hypercalcemia. See O. Kifor et al., J. Clin. Endocrinology & Metabolism 88(1):60-72 (2003). Knowledge of the presence or potential for such antibodies would drastically alter recommended therapy for an individual.

The present methods and kits permit the determination of the development of neutralizing antibodies in a subject, which development need not be an event having immediate adverse consequences for the subject. Based on the determination and monitoring of neutralizing antibodies therapy may be altered or begun that specifically addresses the development of or potential for the development of neutralizing antibodies to a variety of substances.

### III. DISCLOSURE OF THE INVENTION

In one embodiment a method of monitoring the therapeutic inactivating capacity of a subject, said method comprising: a) obtaining a sample from a subject to be monitored; b) assessing said sample for a therapeutic inactivating component specific for a therapeutic agent that has been administered, is being administered or will be administered to said subject, wherein the therapeutic inactivating component binds with said therapeutic agent and interferes with the utility of said therapeutic agent; and c) deciding to initiate, terminate, or adjust the level of administration of said therapeutic agent to said subject based on said assessed therapeutic inactivating component. In a frequent embodiment, the sample is not assessed via plasmon resonance when the therapeutic inactivating component is an antibody. In an occasional embodiment, the therapeutic inactivating component is not an antibody; or when the therapeutic agent is insulin and/or thyroglobulin, the therapeutic inactivating component is not an auto antibody. In a less occasional embodiment, the sample is not assessed via plasmon resonance and/or a delayed solid phase immunologic assay. In another less occasional embodiment, when the therapeutic inactivating component is an auto antibody, such auto antibody is not anti-thyroglobulin and/or it is not assessed via a competitive assay. On occasion, multiple therapeutic agents may be identified and the biological sample is assessed for a therapeutic inactivating component specific for each therapeutic agent.

In frequent embodiments, the present methods are useful for evaluating clinical samples such as human clinical samples. The present methods are equally applicable for a variety of subjects such as subjects within the mammalian genus. Although any of a variety of sample types are useful for monitoring therapeutic inactivating capacity, frequently these samples are blood samples. Blood samples may be whole blood samples, serum samples, plasma derivatives, or assays of any of the whole blood components. Also frequently, urine and other bodily fluid samples may be assayed by the presently described methods.

In one aspect, the subject is afflicted by or believed to be afflicted by a medical condition. Moreover, although not bound by theory, the contemplated therapeutic agents should be useful for treating the medical condition or the underlying symptomology of the medical condition. In frequent embodiments, the therapeutic agent is selected from the group provided in Table 2. In occasional embodiments, the therapeutic agent is selected from the group consisting of a pain management agent, an antipyretic agent, a migraine agent, a prophylaxis agent, an anti-infective agent, an (anti-) inflammatory agent, an (anti-) parasitic agent, a uterine agent, a (anti-) microbial agent, an (anti-) arthritic agent, a gout related agent, a cardiovascular agent, a cancer agent, an immunomodulation agent, a metabolic agent, a musculoskeletal agent, a (anti-)toxicity agent, a dermatologic agent, an ophthalmic agent, an otic agent, a pharyngeal agent, a nasal agent, an HIV or AIDS related agent, an allergy or asthma related agent, an Alzheimer's disease related agent, a diabetes related agent, a glandular disorder related agent, a kidney disease related agent, a liver disease related agent, a mental health related agent, an osteoporosis related agent, a Parkinson's disease related agent, an osteoporosis related agent, a renal bone disease agent, an agent to treat disorders of the parathyroid gland, a sexually transmitted disease related agent, a stroke related agent, a blood or circulatory related agent, an endocrine related agent, a gastrointestinal agent, a neurological agent, and a respiratory agent. On occasion, multiple therapeutic agents are identified and the biological sample is assessed for a therapeutic inactivating component specific for a selection of one or more of the therapeutic agent(s).

A variety of medical conditions are contemplated herein. For example, in one aspect, the medical condition is selected from the group consisting of a pain management related condition, a migraine related condition, an infection, an inflammatory related condition, a urinary related condition, an OB/GYN disorder, an arthritic related condition, a foot related condition, a cardiovascular related condition, a metabolic related condition, a musculoskeletal related condition, cancer, an immunological related condition, a toxicity related condition, a dermatologic related condition, an ophthalmic related condition, an otic related condition, a pharyngeal related condition, a nasal related condition, a blood or circulatory related condition, AIDS, allergy & asthma, Alzheimer's disease, a child specific condition, diabetes, a glandular disorder, kidney disease, liver disease, mental health related condition, osteoporosis, renal bone disease, Parkinson's disease, a sexually transmitted disease, stroke, an endocrine related condition, a gastrointestinal related condition, a neurological related condition, and a respiratory related condition.

In frequent embodiments, a therapeutic inactivating component comprises a therapeutic inactivating antibody or antibody fragment. Also frequently, a therapeutic inactivating component comprises one or more blood or bodily fluid components in a subject that may bind a therapeutic agent, if administered. In one aspect, this binding reduces or eliminates the therapeutic efficacy of the therapeutic agent. In occasional embodiments, a therapeutic agent would be harmful to a subject if administered and the present methods are useful to avoid an adverse reaction to the therapeutic agent. Frequently, the adverse biological reaction comprises the generation of an immune response to the therapeutic agent.

In one aspect of the present methods, the presence or absence of a therapeutic inactivating component is assessed. Frequently, the level of the therapeutic inactivating component is assessed. Although not bound by theory, in general, levels of a therapeutic inactivating component are assessed if the therapeutic inactivating component is present in a sample in more than a nominal and detectable amount. In a related aspect, the therapeutic inactivating component is assessed by a sandwich or competitive assay format. On occasion, the therapeutic inactivating component is assessed by a competitive assay format when the therapeutic inactivating component is not an auto antibody. In occasional embodiments, a delayed solid phase immunologic assay is avoided. Frequently, the therapeutic inactivating component is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), electron transfer assay, nephelometry, flow cytometry assay, chemiluminescence assay, lateral flow immunoassay, u-capture assay, inhibition assay and avidity assay. On occasion, the therapeutic inactivating component is assessed in a homogeneous or a heterogeneous assay format. In a further aspect, the known methods utilized to assess the presence or particular levels of therapeutic inactivating component comprise generally practiced clinical methods, although other methods may suffice for the presently described methods.

Reference to therapeutic agents, hormones, chemical moiety-based therapeutic agents, and natural substances herein includes analogs thereof. In one embodiment, the therapeutic agent may be an agent that treats an immune system disease or disorder, a metabolism disease or disorder, a muscle and bone disease or disorder, a nervous system disease or disorder, a signal disease or disorder, a transporter disease or disorder, a tumor or an infection. In another embodiment, one or more therapeutic agents are available for treating a specific medical condition or category, and the decision to initiate, terminate, or adjust the level of any one of the one or more therapeutic agents is based on said assessed therapeutic inactivating component. Frequently, the therapeutic agent is a chemical moiety comprising a small molecule or a biomolecule. Also frequently, the therapeutic agent comprises a protein or a peptide. In one embodiment, the therapeutic agent is prescription drug or an over the counter drug. Moreover, the present methods contemplate any of a variety of therapeutic agents; for example: LIPITOR® (atorvastatin), EPOGEN® (epoetin alfa), ZEMPLAR® (paricalcitol), RISPERDAL® (risperidone), a calcimemetic, LA SIX® (furosemide), a bisphosphanate, FORTEO® (teriparatide), and numerous others. Moreover, Table 2 provides a listing of some of the contemplated prescribed therapeutic agents. However, one of skill in the art would understand the present methods are generally applicable to a variety of other therapeutic agents.

In another embodiment, the present methods are useful to assess the presence and/or levels of therapeutic inactivating component in a subject that has received an administration of the presently described therapeutic agents. In related embodiments, frequently the subject has received one or more administrations of the therapeutic agent. On occasion, the subject has received one or more courses of administration of the therapeutic agent. Also, on occasion, a subject that had received administration of the therapeutic agent prior to the practice of the present methods, such administration had produced clinically effective results from the therapeutic. Although not bound by theory, the present methods are useful for subjects that had previously received a specific therapeutic agent, including one of a class of therapeutic agents. Occasionally, the present methods are conducted as a part of clinical trial or a drug discovery process. Frequently, the present methods are conducted in a clinical lab.

In another embodiment, the subject has previously and is currently receiving administration of the therapeutic agent, wherein the therapeutic inactivating component is an antibody, and wherein the sample is assessed for the presence or absence of the antibody. Frequently, the subject is monitored over a prolonged period of treatment with the therapeutic agent for the development of the antibody. On occasion, the therapeutic agent is recombinant erythropoietin or an erythropoietin analog.

In a further embodiment the present methods are useful to assess the presence and/or levels of therapeutic inactivating component in a subject receiving administrations of one or more of the presently contemplated therapeutic agents. In another embodiment, the present methods are useful to assess the presence and/or levels of therapeutic inactivating component in a subject that will receive one or more of the presently contemplated therapeutic agents. In yet another embodiment, the present methods are useful to assess the presence and/or levels of therapeutic inactivating component in a subject that has received one or more of the presently contemplated therapeutic agents.

In a frequent embodiment, the present methods are utilized to aid a decision to initiate, terminate, increase, decrease or maintain the administration of a therapeutic agent to a subject based on the assessed therapeutic inactivating component. Frequently, the presence, absence, or related levels of one or more of the therapeutic inactivating components present in a subject are useful to guide the administration of therapeutic agents. Also frequently, a decision for initiating, terminating or adjusting the level of administration of the therapeutic agent to the subject based on the presently provided methods is made by a health care provider or a personnel of a health care management entity, for example, a clinician or a nurse.

Occasionally, in practice, the steps of (a) obtaining and (b) assessing a sample (including embodiments having multiple assessing steps), and the step of (c) deciding treatment based on the results of step (b) can occur at different locations and can be performed by different individuals or entities. Moreover, on occasion, the steps of (a) obtaining and (b) assessing a sample are performed by one individual or entity and the step of (c) deciding treatment based on the results of step (b) is performed by another individual or entity. Also on occasion, each of the steps (a) obtaining a sample, (b) assessing a sample, and (c) deciding treatment based on the results of step (b), are each performed by a the same or different individual or entity at the same or different locations. Thus, any one or combination of the steps of the present methods and embodiments can occur at different locations and be performed by different individuals and/or entities.

In one embodiment a method is provided for determining and/or monitoring a therapeutic protocol for a subject afflicted with an auto antibody specific for a natural substance, said auto antibody developed as a result of said subject having a medical condition that results in an overproduction of said natural substance, said method comprising: obtaining a sample from said subject; assessing said sample for the presence and/or level of said natural substance; c) assessing said sample for the presence of an auto antibody that specifically bind said natural substance; and d) deciding to initiate, terminate, or adjust the level of administration of a therapeutic agent to said subject based on said assessed auto antibody. In an alternative preferred aspect of the above embodiment, the auto antibody developed as a result of therapeutic administration of the natural substance or an analog thereof. On occasion, the auto antibody is specific for a receptor involved in a biological pathway affected by the natural substance. In one embodiment, the auto antibody is not assed via plasmon resonance, and/or a competitive assay. In another embodiment, the natural substance is not insulin and/or thyroglobulin, and/or the auto antibody is not assed via plasmon resonance and/or a competitive assay. Frequently, the therapeutic agent is useful for treating the medical condition or the underlying symptomology of the medical condition. Frequently, the therapeutic agent is selected from the group provided in Table 2. Moreover, frequently the medical condition is a medical condition described or listed herein. In a further embodiment, the presence or absence of the auto antibody is assessed. Frequently, the auto antibody is assessed by a sandwich assay format or an appropriate assay format selected from those provided elsewhere herein.

In another embodiment, the decision for initiating, terminating or adjusting the level of administration of the therapeutic agent to the subject is made by a health care provider or a personnel of a health care management entity. Frequently, the health care provider is a clinician or a nurse. Also frequently, the present methods are conducted in a clinical laboratory. In a further embodiment, the therapeutic agent comprises a compound having a antagonistic biological effect to that normally exhibited by the natural substance.

In one embodiment, the natural substance is parathyroid hormone (PTH). Frequently, the natural substance is erythropoietin. On occasion, the auto antibodies are specific for a receptor involved in a biological pathway affected by the natural substance. In a related embodiment, the natural substance is PTH and the auto antibodies are specific for a receptor involved in a biological pathway affected by PTH. In one aspect of this embodiment, the receptor is a calcium sensing receptor.

In a further embodiment, a method is provided for determining or monitoring a therapeutic protocol for a subject receiving or about to receive administration of a chemical moiety-based therapeutic agent, said method comprising: obtaining a sample from said subject; assessing said sample for a therapeutic inactivating component specific for said chemical moiety-based therapeutic agent that has been administered, is being administered or will be administered to said subject, wherein said therapeutic inactivating component binds with said chemical moiety-based therapeutic agent and interferes with the utility of said therapeutic agent; and deciding to initiate, terminate, or adjust the level of administration of said chemical moiety-based therapeutic agent to said subject based on said assessed therapeutic inactivating component. Frequently, the chemical moiety-based therapeutic agent is a small molecule. Also on occasion, the chemical moiety-based therapeutic agent is a biomolecule. In certain embodiments, the chemical moiety-based therapeutic agent is prescription drug or an over the counter drug. In one aspect, the presence or absence of the therapeutic inactivating component is assessed. As described above, frequently the therapeutic inactivating component is assessed by a sandwich or competitive assay format and/or an another assay format described herein. Frequently, the chemical moiety-based therapeutic agent is useful for treating a medical condition or the underlying symptomology of the medical condition. Moreover, frequently the medical condition is a medical condition described or listed herein.

In another embodiment, the decision for initiating, terminating or adjusting the level of administration of the chemical moiety-based therapeutic agent to the subject is made by a health care provider or a personnel of a health care management entity. Frequently, the health care provider is a clinician or a nurse. Also frequently, the present methods are conducted in a clinical laboratory.

In another embodiment, a method is provided wherein the subject has previously and is currently receiving administration of the chemical moiety-based therapeutic agent, wherein the therapeutic inactivating component is an antibody, and wherein the sample is assessed for the presence or absence of the antibody. In frequently embodiments, the subject is monitored over a prolonged period of treatment with the chemical moiety-based therapeutic agent for the development of the antibody.

In yet another embodiment, a method is provided for monitoring the hormone inactivating capacity of a subject, the method comprising: obtaining a sample from a subject to be monitored; assessing said sample for a hormone inactivating component specific for a hormone, wherein said hormone inactivating component binds with said hormone and interferes with the normal biological activity of said hormone; and deciding to initiate, terminate, or adjust the level of therapeutic administration of said hormone to said subject based on said assessed hormone inactivating component. In one aspect, a reference to a hormone includes analogs thereof, e.g., synthetic analogs. Frequently, the hormone is of the general type normally produced endogenously in the subject. In frequent embodiments, the hormone (or analog thereof) has been administered, or is being administered to the subject as a therapeutic agent. On occasion, the hormone (or analog thereof) will be administered to the subject as a therapeutic agent. In another embodiment, the hormone is recombinant erythropoietin or erythropoietin analog and the hormone inactivating component is an antibody specific for the recombinant erythropoietin or erythropoietin analog. In still another embodiment, the sample is not assessed via plasmon resonance when the hormone inactivating component is an antibody; and when the hormone is insulin and/or thyroglobulin, the hormone inactivating component is not an auto antibody. On occasion, when the hormone inactivating component is an auto antibody the sample is not assessed via a competitive assay. In a further occasional embodiment, the hormone is an agent other than insulin or thyroglobulin. In a further embodiment, the hormone is insulin and the hormone inactivating component is an auto antibody, which auto antibody specifically binds insulin, but wherein the sample is assessed for an increase in the auto antibody that specifically binds insulin, and the increase is a result of exogenous administration of insulin to the subject. In a still further embodiment, the hormone is thyroglobulin and the hormone inactivating component is an auto antibody, which auto antibody specifically binds thyroglobulin, but wherein the sample is assessed for an increase in the auto antibody that specifically binds thyroglobulin, the increase is a result of exogenous administration of thyroglobulin to the subject, and the auto antibody is assessed via means other than a competitive assay.

In another embodiment, a method is provided wherein the subject has previously and is currently receiving administration of the hormone wherein the therapeutic inactivating component is an antibody, and wherein the sample is assessed for the presence or absence of the antibody. In a frequent embodiment, the subject is monitored over a prolonged period of treatment with the hormone for the development of the antibody. In an occasional embodiment, the hormone is recombinant erythropoietin or an erythropoietin analog.

In one embodiment, kits are provided for monitoring therapeutic inactivating capacity of a subject, certain of these kits comprise, for example, a means for assessing therapeutic inactivating component of a sample obtained from a subject to a therapeutic agent that has been administered, is being administered or will be administered to said subject; and instructions for making a decision for initiating, terminating or adjusting the level of administration of said therapeutic agent to said subject based on said assessed therapeutic inactivating component. In frequent embodiments such kits are prepared in accordance with the methods and reagents described herein. In another embodiment, a kit is provided comprising a therapeutic agent; and instructions for monitoring the therapeutic inactivating capacity of a subject to said therapeutic agent.

Frequently, such kits further comprise a means for obtaining a sample from a subject to be monitored. Also frequently, the subject is afflicted by or believed to be afflicted by a medical condition, and wherein the therapeutic agent is useful for treating the medical condition or the underlying symptomology of the medical condition and is selected from those described and listed herein. Frequently, the therapeutic agent is selected from the group provided in Table 2.

Methods and kits of the present description frequently utilize an unhindered therapeutic agent, unhindered hormone, unhindered natural substance or unhindered chemical moiety-based therapeutic agent and a low molecular weight label bound to the unhindered therapeutic agent, unhindered hormone, unhindered natural substance or unhindered chemical moiety-based therapeutic agent, respectively. In frequent embodiments, a sample is assessed according to the present methods for a therapeutic inactivating component, a hormone inactivating component, or an auto antibody that is specific for a therapeutic agent/chemical moiety-based therapeutic agent, hormone, or natural substance, respectively. Frequently, the sample is assessed via steps comprising: contacting the sample containing the therapeutic inactivating component, if any, with the therapeutic agent to form a reaction mixture, wherein the therapeutic agent comprises an unhindered form of the therapeutic agent bound by a low molecular weight label, and wherein the therapeutic inactivating component, if any, binds the unhindered, labeled therapeutic agent to form a labeled therapeutic inactivating component complex; separating the labeled therapeutic inactivating component complex from the reaction mixture; and assessing the separated labeled therapeutic inactivating component complex. In frequent embodiments, the presence and/or concentration of the separated labeled therapeutic inactivating component, if present, is assessed. Also frequently, the proportion of the molecular weight of the low molecular weight label, versus the molecular weight of the unhindered therapeutic agent, unhindered hormone, unhindered natural substance or unhindered chemical moiety-based therapeutic agent, comprises less than about 50%. Frequently, the low molecular weight label comprises a radioactive label, e.g., Iodine-125, or a chemiluminescent label, e.g., luminol, although other low molecular weight labels are contemplated.

In a preferred aspect, the molecular weight of the low molecular weight label bound to the unhindered therapeutic agent, unhindered hormone, unhindered natural substance or unhindered chemical moiety-based therapeutic agent totals less than 40%, less than 30%, less than 20%, or less than 10% of the weight of the unhindered therapeutic agent, unhindered hormone, unhindered natural substance or unhindered chemical moiety-based therapeutic agent. In another preferred aspect, the molecular weight of the low molecular weight label bound to the unhindered therapeutic agent, unhindered hormone, unhindered natural substance or unhindered chemical moiety-based therapeutic agent totals between about than 50% and 1% of the weight of the unhindered therapeutic agent, unhindered hormone, unhindered natural substance or unhindered chemical moiety-based therapeutic agent. In a particularly preferred aspect, the molecular weight of the low molecular weight label bound to the unhindered therapeutic agent, unhindered hormone, unhindered natural substance or unhindered chemical moiety-based therapeutic agent totals between about 1% to about 10%, or about 3% of the weight of the unhindered therapeutic agent, unhindered hormone, unhindered natural substance or unhindered chemical moiety-based therapeutic agent. In another aspect, the molecular weight of the low molecular weight label bound to the unhindered therapeutic agent, unhindered hormone, unhindered natural substance or unhindered chemical moiety-based therapeutic agent totals between about 1% to about 10% of the weight of the unhindered therapeutic agent, unhindered hormone, unhindered natural substance or unhindered chemical moiety-based therapeutic agent.

### IV. BRIEF DESCRIPTION OF THE DRAWINGS

FIG. 1 depicts three versions of an Assay of human IgG/M against a therapeutic agent comprising PTH. FIG. 1 includes FIGS. 1a-c. Although not bound by theory, the therapeutic agent can alternatively be a natural substance, a naturally occurring therapeutic, a hormone, a receptor and/or a chemical moiety based therapeutic, among others.

FIG. 1a depicts an ELISA utilizing indirect anti-human IgG/M. In this assay a sample of patient serum/plasma is introduced to a well [1] coated with synthetic PTH [2] (alternatively a synthetic PTH coated bead is added to the serum/plasma) (i.e., solid phase). The human anti-PTH [3] binds to the PTH and then a labelled goat anti-human IgG (alternatively labelled goat anti-human IgM or labelled goat anti-human IgG/IgM) [4, 5] is added. The solid phase is then washed and assessed for bound label.

FIG. 1b depicts second antibody precipitation. A basic description of the depicted process is as follows: Labelled synthetic PTH [6, 7] (e.g., Iodine-125, luminol or another label) is added to a sample of serum/plasma. Human IgG [8] (alternatively human IgM or human IgG/IgM), if present in the sample, then binds with the labelled synthetic PTH. Precipitating antibody [9] (e.g., goat anti-IgG, goat anti-IgM or goat anti-IgG/IgM) is added to the sample to bind the human IgG and cause the precipitation of the human IgG. After formation of the precipitate, the sample is centrifuged, the supernatant is decanted and the precipitate pellet is assessed for a signal.

FIG. 1c depicts the use of capture beads to immobilize PTH and labelled anti-human IgG/M for detection. The capture beads [10] bound with goat anti-PTH [11] are added to a synthetic PTH sample [12] to attach the PTH [12] to the capture bead solid phase [10]. A sample of serum/plasma is then added to the PTH bound capture beads. Human anti-PTH [13], if present, binds to the solid phase bound PTH. Labelled anti-human antibody [14, 15] (comprising, for example, labelled goat anti-human IgG, goat anti-human IgM and/or goat anti-human IgG/IgM) is then added to the mixture. The mixture is then incubated and the solid phase is washed and assessed for bound label.

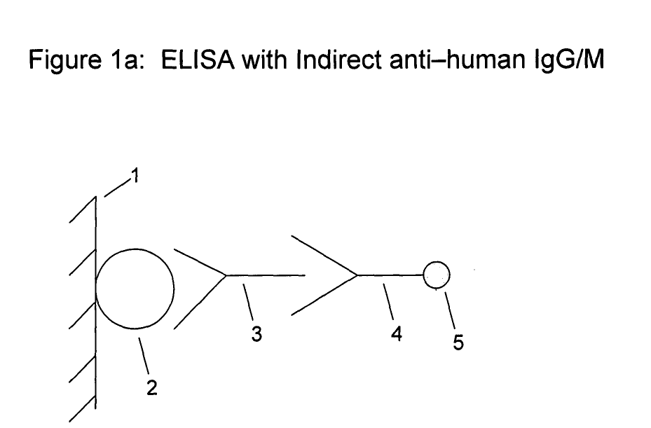

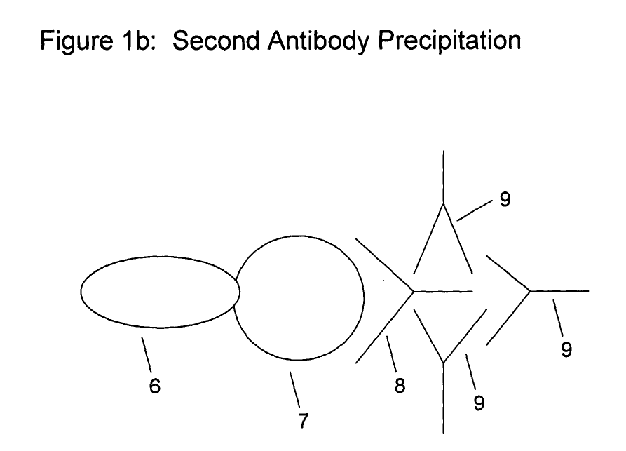

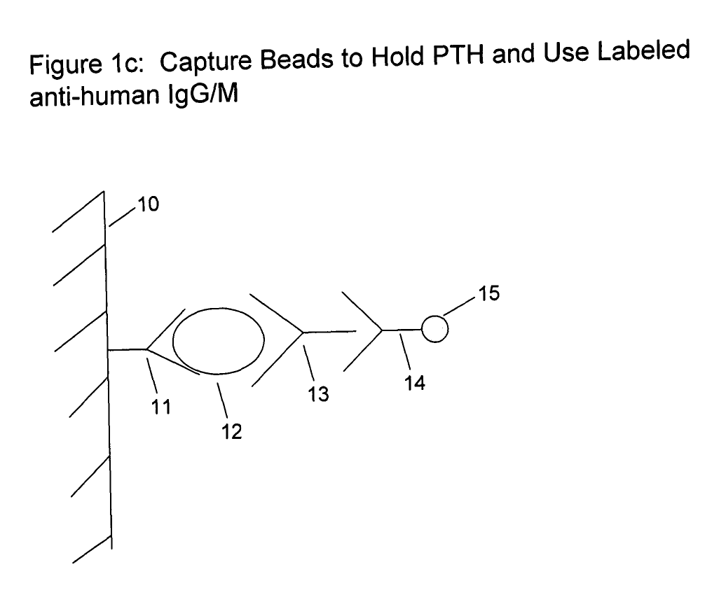

### V. DETAILED DESCRIPTION OF THE INVENTION

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, “afflicted” as it relates to a disease or disorder refers to a subject having or directly affected by the designated disease or disorder.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term “subject” is not limited to a specific species or sample type. For example, the term “subject” may refer to a patient, and frequently a human patient. However, this term is not limited to humans and thus encompasses a variety of mammalian species.

As used herein, the term “terminate” refers to ending a process, method or any other assorted scheme described herein, and also may indicate that a process, method or any other assorted scheme is not to begin or commence. For example, as used herein, a phrase indicating that the use of a therapeutic agent is to be terminated could refer to both ending an ongoing use or not beginning to use the therapeutic agent. Given the circumstances present when the use of such therapeutic agent should be terminated, one of skill in the art would understand the context as applying to those receiving the therapeutic agent versus those that are not.

As used herein, “macromolecule” refers to a molecule that, without attaching to another molecule, is capable of generating an immune response resulting in an antibody that specifically binds to the macromolecule.

As used herein, “chemical moiety-based therapeutic agent” refers to a chemical agent including small molecules, organic and inorganic molecules, among others. Such chemical moiety-based therapeutic agents are not intended to encompass proteins or peptides.

As used herein, “small molecule” refers to a molecule that, without forming homo-aggregates or without attaching to a macromolecule or adjuvant, is incapable of generating an immune response resulting in an antibody that specifically binds to the small molecule. Preferably, the small molecule has a molecular weight that is about or less than 10,000 Daltons. More preferably, the small molecule has a molecular weight that is about or less than 5,000 Daltons.

As used herein, “inorganic molecule” refers to a molecule that does not contain hydrocarbon group (s).

As used herein, “organic molecule” refers to a molecule that contains hydrocarbon group (s).

As used herein, “biomolecule” refers to an organic compound normally present as an essential component of living organisms.

As used herein, “therapeutic inactivating component” or “TIC” refers to a moiety that interferes with the intended mode of action of a therapeutic agent. Frequently, this interference may comprise a complete inhibition of the intended mode of action, however other results are contemplated. For example, a therapeutic inactivating component may reduce or eliminate the efficacy, neutralize, counteract or reverse the intended mode of action of a therapeutic agent. Except for limitations provided herein limitation, a therapeutic inactivating component may comprise an antibody that binds a therapeutic agent comprising a protein or peptide.

As used herein, “medical condition” refers to any of a variety of diseases or disorders or their associated symptomology. Frequently, the present disclosure refers to a medical condition as falling within a subset of disorders or their associated symptomology which may be treated through the use of a therapeutic agent. Medical conditions include known broad therapeutic categories with examples comprising pain management related conditions, migraine related conditions, infections, inflammatory related conditions, urinary related conditions, OB/GYN disorders, arthritic related conditions, foot related conditions, cardiovascular related conditions, metabolic related conditions, musculoskeletal related conditions, cancer, immunological related conditions, toxicity related conditions, dermatologic related conditions, ophthalmic related conditions, otic related conditions, pharyngeal related conditions, nasal related conditions, blood and circulatory related conditions, AIDS, allergy & asthma, Alzheimer's disease, children's conditions, diabetes, glandular disorders, kidney disease, liver disease, mental health, osteoporosis, Parkinson's disease, sexually transmitted disease, stroke, endocrine related conditions, gastrointestinal related conditions, neurological related conditions, and respiratory related conditions. One of skill in the art would understand that the above categories include some overlap, by way of non-limiting example, one such medical condition could be an immunological condition and a respiratory condition as well. Other specific medical conditions may include further and more complex overlap of one or more categories. Moreover, specific therapeutic categories falling within one or more of these categories are clearly contemplated. See Alfred Goodman Gilman et al., THE PHARMACOLOGICAL BASIS OF THERAPEUTICS (9th ed. 1996).

As used herein, “therapeutic agent” or “TA” refers to any of a variety of chemical, biological or mechanical agents used to treat or modulate a medical condition. Moreover, as used herein “therapeutic agent” further refers to analogs of the described therapeutic agents. For example, a therapeutic agent as used herein may refer to a prescription drug. Therapeutic agents include, but are not limited to, prescription or over the counter drugs, small molecules, biomolecules, pain management agents, analgesics, antipyretic agents, migraine agents, prophylaxis agents, recombinant protein agents, anti-infective agents (e.g., anti-virals, anti-protozoals, anti-bacterials, anti-malarials and anti-fungals), (anti-) inflammatory agents, parasitic agents, uterine agents, quinolones, sulfonamides, cephalosporins, macrolides, azalides, beta-lactam antibiotics, penicillins, tetracyclines, microbial agents, arthritic agents, gout agents, cardiovascular agents (e.g., anti-anginals, anti-arrhythmics, hematologic agents, heart failure agents, hypertensive therapy, diuretics, hypolipodemic agents), cancer agents, immunomodulation agents, metabolic agents, musculoskeletal agents, (anti-) toxicity agents, dermatologic agents, ophthalmic agents, otic agents, pharyngeal agents, nasal agents, AIDS and HIV related agents, allergy & asthma related agents, endocrine related agents (e.g., hormones, contraceptives and hypoglycemics), gastrointestinal agents (e.g., ulcer agents, digestant agents, diarrhea agents, nausea agents, and emesis therapy agents), neurologic agents (e.g., neuromodulators, neuromuscular agents, neurohormones, behavior modifiers, anti-psychotics, and anxiolytics), Alzheimer's disease related agents, antidepressants, diabetes related agents, glandular disorder related agents, kidney disease related agents, liver disease related agents, mental health agents, osteoporosis related agents, renal bone disease related agents (e.g., Vitamin D, a Vitamin D analog, a calcimimetic, a lanthanum containing agent, calcium supplement treatment, a parathyroid hormone antagonist, a parathyroid hormone agonist, phosphate, a calcililetic, EDTA, or a calcium binding agent), Parkinson's disease related agents, sexually transmitted disease related agents, stroke related agents, blood and circulatory related agents, endocrine related agents, gastrointestinal agents, neurological agents, and respiratory agents (e.g., antihistamines, bronchodilators, anti-anaphylactic agents, and anti-asthmatic agents). Although a listing of the several contemplated therapeutic agents is included herein, except for express limitations provided herein, such listing is not intended to be limiting of the presently described methods & kits.

In one embodiment, the contemplated therapeutic agents may act on synaptic and neuroeffector junction sites. In another embodiment, the therapeutic agents may be endocrine related therapeutics such as hormones, contraceptives and hypoglycemics. In a further embodiment, the therapeutic agents may be gastrointestinal therapeutics such as ulcer, digestant, diarrhea, nausea, emesis therapy. In another embodiment, the therapeutic agents may be neurologic therapeutics such as anticonvulsant agents, sedatives, Alzheimer's agents, anti-Parkinson agents, musculoskeletal agents, antidepressants, behavior modifiers, anti-psychotics, anxiolytics, etc. In yet another embodiment, the therapeutic agents may be respiratory therapeutics such as antihistamines, bronchodilators, anti-anaphylactic agents, and anti-asthmatic agents.

One of skill in the art would understand that the above therapeutic agent categories include some overlap, by way of non-limiting example, one such therapeutic agent could be an immunomodulation agent and a microbial agent as well. Other specific therapeutic agents may include further and more complex overlap of one or more categories. Moreover, specific therapeutic agents falling within one or more of these categories are clearly contemplated. Examples of some of the currently contemplated therapeutic agents may be found in Table 2 herein. See also Alfred Goodman Gilman et al., THE PHARMACOLOGICAL BASIS OF THERAPEUTICS (J G Hardman et al. eds., 9th ed. 1996). Moreover, information regarding the types of contemplated therapeutic agents may be located, for example, on the publicly accessible Physicians Desk Reference Website on the World Wide Web at “pdrhealth.com/drug_info/index.html.”

As used herein, “antibody” is used in the broadest sense. Therefore, an “antibody” can be naturally occurring or man-made such as monoclonal antibodies produced by conventional hybridoma technology and/or a functional fragment thereof. Antibodies of the present invention comprise monoclonal and polyclonal antibodies as well as fragments containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, the term “specifically binds” refers to the specificity of an antibody such that it preferentially binds to a defined target. Recognition by an antibody of a particular target in the presence of other potential targets is one characteristic of such binding. Specific binding of the presently contemplated antibodies to particular PTH, EPO and other targets is measured through known methods utilizing the tools provided herein.

B. Description of Preferred Embodiments

In one aspect, the present disclosure encompasses the idea that therapeutic decisions may be enhanced or rendered more accurate though a procedure that routinely investigates for one or more particular therapeutic inactivating components (TICs) in a subject prior to prescribing a therapeutic agent. In particular subjects, a therapeutic inactivating component may be present that reduces the efficacy or completely inactivates a therapeutic agent such that no therapeutic benefit is achieved through its administration. In other subjects, an adverse biological reaction may be generated through the administration of a particular therapeutic agent. Such reactions may be specific or general adverse biological reactions to the therapeutic agent or a component of the agent.

In certain subjects that have a therapeutic inactivating component that reduces the efficacy or eliminates the biological activity of a therapeutic agent or class of therapeutic agents, several benefits may be realized through the practice of the presently described methods. In one aspect, the most effective therapeutic agent for the subject may be selected prior to administration. In this aspect, the concept of effectiveness may be gauged via reference to the presence and/or level of a therapeutic inactivating component. For example, given a particular subject having a particular medical condition, a set of multiple therapeutic agents may be available to treat the specific medical condition. Upon practice of the present methods, the set may be narrowed to one or more therapeutic agents due to the determination that a therapeutic inactivating component is present in the subject that is specific for one or more of the therapeutic agents in the set. Thus, prior to administration of a particular therapeutic agent, it would be determined which of those known or available therapeutic agents are more likely to effect the intended therapeutic purposes. Based on the presently disclosed methods, an artisan would understand that such a procedure would be equally applicable to, and beneficial for, subjects that have previously or are currently receiving administration of a particular therapeutic agent. Evaluation of these subjects under the present methods provides a means to avoid future administration of a therapeutic agent to a subject if a therapeutic inactivating component specific for that agent is present in the subject.

In certain subjects where an adverse biological reaction may be generated through the administration of a particular therapeutic agent or class of therapeutic agents, several benefits may be realized through the practice of the presently described methods. In one aspect, a relatively safe therapeutic agent may be selected prior to administration to a subject. Although not bound by theory, an adverse reaction may not always be predictable based on the methods discussed herein. However, as discussed herein, particular embodiments of the present methods provide for assessment of a sample prior to administration of a therapeutic agent. Such an assessment would recognize the presence of a therapeutic inactivating component in a subject's sample and thus indicate that the subject therapeutic agent should not be administered. Thus, surprisingly, the possibility of an adverse reaction can be averted for particular subjects (and therapeutic agents) providing a degree of predictability of an adverse reaction if a therapeutic inactivating component is present which is specific for the therapeutic agent in that patient. The present compositions and methods are useful to avoid development of an adverse reaction to a therapeutic agent. Thus, prior to administration of a particular therapeutic agent, it can surprisingly be determined which of those known or available therapeutic agents would be more likely to effect an adverse biological response. Based on the presently disclosed methods, such a procedure is equally applicable to, and beneficial for, subjects that have previously or are currently receiving administration of a particular therapeutic agent. Evaluation of these subjects under the present methods provides a means to avoid future administration of a therapeutic agent to a subject if a therapeutic inactivating component specific for that agent is present in the subject.

In practice, the therapeutic agent may comprise any of a variety of compounds and the therapeutic inactivating component is the result of an immune reaction to an administered therapeutic agent. The immune reaction can take one of several forms but generally comprises an antibody response specific for the therapeutic agent. Moreover, an antibody that “recognizes” the therapeutic agent may set in motion an immune response analogous to the way that the subject's body would normally react to a foreign pathogen. For example, the immune response may take to form of a neutralization reaction, opsonization and/or complement activation. See, e.g., C. A. Janeway, Jr., et al., IMMUNOBIOLOGY (2001). The immune reaction could also take the form of an autoimmunity reaction. See id.

In one embodiment, one or more therapeutic agents are available for treating the medical condition, and the decision to initiate, terminate, or adjust the level of any one of the one or more therapeutic agents (TAs) is based on said assessed therapeutic inactivating component. For example, a sample may be evaluated for the presence of one or more therapeutic inactivating components known to be specific for the contemplated TAs. Any one TA may be ruled out for therapeutic use based on a positive indication that a therapeutic inactivating component exists in a sample that is specific for the TA. Thereafter or concurrently, the level of the therapeutic inactivating component, if present, may be assessed to determine whether TA dosage or concentration should be modified if the TA is still therapeutically indicated, even in light of the presence of a therapeutic inactivating component. Thus, the present methods are useful to modify a recommended TA dosage based on the presence and/or level of one or more therapeutic inactivating components in a sample. While not being bound by theory, certain therapeutic inactivating components may be present in a sample that would not render therapeutically ineffective an entire dosage of a TA. In one embodiment, the present methods are useful to identify one or more TAs that have an increased likelihood of therapeutic efficacy based on the measured therapeutic inactivating component presence and/or level.

In one aspect, the therapeutic inactivating component, if present, may be specific for a therapeutic agent or class of therapeutic agents. The specificity of the therapeutic inactivating component refers to the general effect of the therapeutic inactivating component on the therapeutic agent. If a therapeutic inactivating component is present but does not inhibit the biological activity of, or produce an adverse biological reaction to, the TA, then the therapeutic inactivating component is not considered as specific for that TA. For example, any given subject may have a variety of therapeutic inactivating components, however, given the medical condition of the subject, only a selection of TAs may be useful for treatment of the medical condition. Thus, the relevant therapeutic inactivating components in this example are those that affect the biological activity of, or produce an adverse biological reaction to, the specific TAs for that medical condition. Accordingly, in one particularly preferred embodiment, there is a defined relationship between the medical condition, the therapeutic inactivating components and the TAs. In an alternative embodiment, the relationship between the medical condition, the therapeutic inactivating components and the TAs is specific. For example, in such a specific relationship, the subject is afflicted with a certain medical condition for which a finite set of TAs are known as therapeutically indicated for treatment therefor. Based on this finite set of TAs, the subject may be tested for the presence of therapeutic inactivating components that are specific for (i.e., inhibit the biological activity of, or produce an adverse biological reaction to) any one or more of the TAs.

The present methods and kits are useful for subjects that have received administration of a specific TA, are currently receiving a specific TA, and yet to receive a specific TA. In one embodiment, the subject has previously received, or is currently receiving, administration of a TA or one of a class of TAs, and a therapeutic inactivating component has been produced in the subject at a detectable level as a result of the previous administration of the TA. In another embodiment, the subject has previously received, or is currently receiving, administration of a TA or one of a class of TAs, and a therapeutic inactivating component specific for the TA was present previously, but not recognized or tested for. The present methods also encompass the embodiment wherein the subject has yet to receive the administration of a TA or one of a class of TAs, and a therapeutic inactivating component specific for the TA is present and/or will be generated upon administration of the TA. Therapeutic decisions are possible based on the determination that a therapeutic inactivating component specific for a contemplated TA or class of TAs is present. Therapeutic decisions are also possible in light of the present methods based on the measured presence and level of a therapeutic inactivating component in a sample. While not being bound by theory, therapeutic decision are multi-factoral including evaluation of the medical condition, the TAs useful for treating the condition of the symptomology related to the condition, and the presence and/or level of a therapeutic inactivating component, among other factors. Based on the present methods, the level or concentration of the TA administered may be altered corresponding to the measurement of the presence and/or level of a therapeutic inactivating component. The present methods are also useful to select one or more TAs that have a higher likelihood of therapeutic efficacy for a medical condition in light of the presence and/or level of a therapeutic inactivating component in a subject.

Therapeutic decisions are generally made by health care practitioners or personnel of a health care management entity. Frequently, therapeutic decisions are made by a clinician or nurse. Correspondingly, the methods of the present invention may be readily practiced by a clinical laboratory.

A given therapeutic inactivating component may be specific for the whole therapeutic agent or only a portion of the TA. Nevertheless, for a therapeutic inactivating component to be specific for the TA as described herein, the therapeutic inactivating component must either affect the intended biological activity of the TA or result in an adverse biological activity to the TA. In one aspect the therapeutic inactivating component may be specific for only a portion of the TA. For example, the TA may be a protein and the therapeutic inactivating component may be an antibody that binds the therapeutic inactivating component, thus presenting an immune reaction to the therapeutic agent. In one aspect, the therapeutic inactivating component need not bind an active site of the TA, as long as the therapeutic inactivating component interferes with the intended activity of the TA. For example, the TA may be a protein comprised of 84 amino acids and having a specific epitope ranging from amino acids 7-15 which is instrumental for the intended activity of the therapeutic protein. The therapeutic inactivating component may bind to the protein outside of this epitope, but will still be a therapeutic inactivating component as contemplated herein if this binding interferes with the intended therapeutic activity of the TA.

Therapeutic agents may elicit an undesired immune response specific thereto in a variety of circumstances. Frequently, such immune responses are a product of the purification, formulation and/or storage of the therapeutic agent. Structural properties of the therapeutic agents may also play a role, such as sequence variation and glycosylation. Other factors include contaminants and impurities, downstream processing, route of application, dose and length of treatment, a variety of biological characteristics of the subject receiving or to receive the therapeutic agent, among other factors. A non-limiting selection of therapeutic agents that have a history of inducing an immune response thereto is the following: Interferon alpha 2, interferon beta, GM-CSF, erythropoietin, interleukin-2, growth hormone, GnRH, desoxyribonuclease, HCNTF, GM-CSF/IL3, TNFR55/IgG1, TNFR70/IgG1, Denileukin difitox, insulin, streptokinase, staphylokinase, ADA, calcitonin, thrichosantin, interferon alpha consensus, methionyl human growth hormone, Factor VIII, glucocerebrosidase, hCG, pegylated interferon alpha-2, and pegylated macrophage-derived growth factor (MDGF).

Although the immune reaction developing from the administration of a therapeutic agent may vary, often such reactions are broken into categories based on the source of the therapeutic agent, e.g., derived/obtained from plant, microbial or animal sources. As such, the therapeutic inactivating component may vary to include, e.g., neutralizing antibodies or binding antibodies. Although not limited by theory, neutralizing antibodies decrease efficacy of the administered therapeutic agent and occasionally neutralize a native protein which the therapeutic agent was provided to supplement or replace. Such neutralization of the native protein is significantly detrimental to the subject, for example, because the native protein would not longer achieve its intended function. In the case of erythropoietin, such neutralization would cause the cessation of new red blood cell production therapeutic inactivating components can reduce or eliminate efficacy for insulin, Interferon alpha 2, interferon beta, interleukin-2, GnRH, TNFR55/IgG1, Denileukin difitox, streptokinase, staphylokinase, ADA, calcitonin, Factor VIII, and hCG, among others. Moreover, therapeutic inactivating components have been shown to induce neutralization of native protein as a result of the exogenous administration of erythropoietin and MDGF to particular subjects. See, e.g., Ponnappan, S., et al., Hybridoma 19(5):355-61 (2000); Dybedal, I., Jacobsen, S. E., Blood 86(3):949-57 (1995); Ben Ghanem A, et al, Hybridoma 12(5):599-608 (1993).

A variety of assays are useful for assaying a sample for a therapeutic inactivating component. For example, without limitation, monitoring for a therapeutic inactivating component to recombinant erythropoietin can take the form of a radio immune precipitation assay, an ELISA, a BIAcore assay, or a bioassay. See, e.g., CURRENT PROTOCOLS IN IMMUNOLOGY (John E. Coligan, et al. eds. 2000); and CURRENT PROTOCOLS IN MOLECULAR BIOLOGY (Frederick M. Ausubel, et al. eds. 2000). Such assays are useful regardless of the type or source of recombinant erythropoietin, e.g., EPOGEN® (Amgen, Inc., Thousand Oaks, Calif.), EPREX® (Johnson & Johnson Corp., New Brunswick, N.J.), NEORECORMON® (F. Hoffmann-La Roche Ltd., Basel, Switzerland), ARANESP® (Amgen, Inc., Thousand Oaks, Calif.), PROCRIT® (Johnson & Johnson Corp., New Brunswick, N.J.), or ESPO® (Kirin Brewery Co, Ltd.).

In one embodiment, the therapeutic agent is an antimicrobial agent and the therapeutic inactivating component is an enzyme that inactivates the therapeutic agent. For example, the therapeutic agent is penicillin and the therapeutic inactivating component is penicillinase comprising any of various enzymes that are produced by certain bacteria (e.g., Neisseria gonorrhoeae), including most strains of staphylococci, and that hydrolyze and inactivate penicillin. In another embodiment, the therapeutic agent may be or contain a component that chelates with a therapeutic inactivating component comprising metal ions, which prevents absorption of the therapeutic agent. For example, the therapeutic agent may contain ethylenediaminetetraacetic acid, edatate di sodium, or edatate calcium disodium which could cheal ate with metal ions such as zinc manganese, iron, lead. Or, the therapeutic agent contains diethylenetriaminepentaacetic acid that chealates with a heavy metal ion. Other therapeutic agents may contain compounds such as dimercaprol (2,3-dimercaptopropanol), succimer (2,3-dimercaptosuccinic acid), penicillamine (dextro-β,β-dimethylcysteine), trientine (trithylenetetramine dehydrochloride), deferoxamine (deferoxamine mesylate), among others that will chealate with one or more heavy metal ions.

As described above, except for limitations provided herein, the present invention is not intended to be limiting in terms of the potential therapeutic agents contemplated. However, a measurable therapeutic inactivating component must generally exist which is specific for the present therapeutic agents. Frequently, the present methods are useful to measure physiological amounts of the therapeutic inactivating component(s). The present methods are useful to determine whether a therapeutic inactivating component is present in a sample or absent from a sample. In one further embodiment, the present methods are useful to determine the level of one or more therapeutic inactivating components in a sample.

A variety of assays are useful to determine whether a therapeutic inactivating component is present in a sample and to determine the level of a therapeutic inactivating component in a sample, if present. For example, the present methods contemplate the use of sandwich format to determine the presence or level of a therapeutic inactivating component. On occasion, the therapeutic inactivating component is assessed by a competitive assay format when the therapeutic inactivating component is not an auto antibody. In occasional embodiments, a plasmon resonance assay is avoided. In one embodiment, the therapeutic inactivating component is assessed in a homogeneous or a heterogeneous assay format. In another series of embodiments, the present methods and kits provide for the use assay formats selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, u-capture assay, inhibition assay and avidity assay.

Monoclonal antibody immunodiagnostics are currently utilized to aid in diagnosing and treating cancers expressing particular proteins. For example, PROSTASCINT® (Capromab Pendetide) (available from Cytogen Corp., Princeton, N.J.), a murine monoclonal antibody based diagnostic produced from a human prostate carcinoma cell line specifically binds a high percentage of prostate specific membrane antigen (PMSA), which is frequently highly expressed in malignant prostate cells. Due to the nature of the PROSTASCINT® as a murine monoclonal antibody, the potential exists for the induction of undesired human anti-mouse IgG production in patients to whom PROSTASCINT® is administered. Detectable serum levels of HAMAs can alter the clearance and tissue biodistribution of MAbs. Although not bound by theory, the development of persistently elevated serum HAMA levels could compromise the efficacy of agents based on murine antibodies. When considering the administration of murine antibody based diagnostics or therapeutics to subjects that have previously received other murine antibody-based products, one should be aware of the potential for assay interference, increased clearance, and altered biodistribution, which may interfere with the quality or sensitivity of the administered agent. Thus, prior to administration of murine antibody based reagents, it should be determined whether the patient has previously received such products and/or whether the subject otherwise has circulating HAMAs. For example, in one embodiment a diagnostic comprising a murine based antibody (e.g., ONCOSCINT®) specific for a protein/receptor that is differentially over expressed in ovarian cancer is injected into women suspected of having ovarian cancer, if ovarian cancer is present the diagnostic will bind to the protein/receptor and it can be imaged. In another example, the diagnostic or therapeutic contains a murine based antibody specific for a protein/receptor that is differentially over expressed in a cancer which is administered to a subject suspected of being afflicted with a cancer that expresses the specific protein or receptor. One of skill in the art would understand that the specific receptor, protein or cancer is important, and that the nature of the basis of the diagnostic or therapeutic, being a murine based antibody, is of particular importance in certain embodiments.

In another embodiment, a subject is tested for the presence of one or more HAMAs and the therapeutic or diagnostic course is determined based on the circulating concentration of HAMAs present in the subject. For example, the present methods allow the determination of whether given a particular circulatory concentration of HAMAs is indicative of whether initial or continued treatment with a particular therapeutic agent will be ineffective (i.e., as the HAMA would bind and inactivate the antibody agent) and/or dangerous (i.e., risk of anaphylaxis). Such a concentration may comprise a circulating concentration of about 300 ng/ml to about 400 ng/ml of HAMA within a subject that is specific for the therapeutic/diagnostic to be administered.

PTH peptides are utilized as pharmaceuticals to treat a bone turnover related disease or disorder in a subject (e.g., FORTEO® (comprising PTH₁₋₃₄) available from Eli Lilly, Indianapolis, Ind.). Frequently, the active component of the PTH pharmaceutical frequently comprises at least PTH₁₋₃₄ peptide and the subject is a patient afflicted with a bone turnover related disorder, such as osteoporosis. In one embodiment of the present disclosure, the active component of the PTH pharmaceutical frequently comprises at least PTH₁₋₈₄ peptide and the subject is a patient afflicted with a bone turnover related disorder, such as adynamic low bone turnover disease. In a related aspect, therapy to increase the bone turnover rate in a subject can often comprise administering PTH agonist, phosphate, calcililetic, PTH, EDTA, calcium binding agents or stimulating PTH production or a combination thereof. See e.g., Goodman W G, Turner S A, Adv. Ren. Replace Ther. 2002 July;9(3):200-8 (calcimimetic agents); Parthemore J G, et al., J. Clin. Endocrinol. Metab. 1978 August;47(2):284-9 (EDTA). In another embodiment of the present disclosure, the subject is afflicted with adynamic low bone turnover disease or osteoporosis and the PTH pharmaceutical PTH₁₋₈₄ peptide or PTH₁₋₃₄ peptide or cyclase active PTH peptides within these bounds.

In one embodiment of the present disclosure, the presence of a therapeutic inactivating component to a PTH pharmaceutical is determined in a subject. For example, 3/16″ polystyrene capture beads coated with 2 micrograms each of affinity purified goat anti human PTH 37-84 (Scantibodies Laboratories, Santee, Calif.) are combined with 1 ng of PTH₁₋₈₄ peptide in 3001 μl of buffer and protein, e.g., PBS buffer containing 1% BSA, pH 7.4, and allowed to incubate overnight. The beads are then washed with 2 mL of wash solution comprising phosphate buffered saline with sodium azide 1.5% (w/v) and detergent and combined with 10011 of plasma from a test subject and 200 μl of PBS buffer containing 1% BSA. This combination is then incubated overnight at room temperature. Iodinated affinity purified goat anti human IgG is then contacted with the beads. The beads are subsequently washed three times with 2 mL of wash solution comprising phosphate buffered saline with sodium azide 1.5% (w/v)-and detergent and counted with a gamma counter to determine the presence of antibodies specific for PTH₁₋₈₄. See FIG. 3.

In one embodiment, subjects are selected and samples are assayed to determine the presence and/or circulating concentrations of auto antibodies against PTH. The PTH molecule in this embodiment is generally endogenously produced. The presence of auto antibodies against PTH may indicate a natural adaptive mechanism developed to buffer the effects of a malady in a subject that results in the over-production of PTH. Alternatively, the malady exists at the level of production of such auto antibodies, without the corresponding over-production of PTH. In either circumstance, the presence of auto antibodies specific for PTH has definite implications for PTH related therapeutics useful for a subject. For example, auto antibodies specific for PTH, if present, will result in decreased levels of calcium production and decreased bone turnover. Such effects would not typically be effected through a PTH antagonist pathway, but would rather result from the decreased effect of circulating PTH. Where PTH would normally increase calcium production and bone turnover, the decreased efficacy thereof will generally result in both of these levels lowering or remaining stagnant. In individuals suspected of having high bone turnover and/or PTH production and the corresponding physiological effects, information about the presence of auto antibodies against PTH will aid the selection of an appropriate therapeutic agent. In such subjects, Vitamin D is frequently prescribed to suppress PTH production. For these individuals, the knowledge of auto antibodies against PTH will allow for the selection of, at the very least, a lowered concentration of Vitamin D. The administration of excessive dosages of Vitamin D to suppress PTH can have profound detrimental consequences to patients through, for example, hypercalcemia, adynamic low bone turnover, as well as vascular and soft tissue calcification. In addition, knowledge of the presence of auto antibodies against PTH, in addition to potential characterization of the auto antibodies, can allow for the use of a recombinantly produced PTH based therapeutic that will avoid detection (and inactivation) by the auto antibodies, while affecting the desired PTH related biological pathway.

In another embodiment, auto antibodies are present in a subject that are specific for an extracellular calcium sensing receptor. Determination of the presence or potential for such auto antibodies indicates that administration of calcium in these subjects is not therapeutically indicated. Rather, therapy comprising administration of a PTH antagonist such as PTH₇₋₈₄ to counter potential or actual overproduction of PTH₁₋₈₄ would be more appropriate in such subjects. See, e.g., U.S. application Ser. No. 09/323,606, filed Jun. 1, 1999.

An assay of a larger patient population (e.g., about 500 or more patients and more preferably, more than about 1000 patients) will aid in determining the frequency of a hormone inactivating component (i.e., auto antibodies) to native PTH and to determine variations of therapies in light of the presence of the hormone inactivating components. The present methods are also useful to assay the presence and concentrations of antibodies for any of a variety of other PTH peptide pharmaceuticals. Such PTH peptide pharmaceuticals may comprise PTH fragments such as PTH₇₋₈₄, and others of the sort, which agonize or antagonize normal biological activity of PTH in a subject.

In a less preferred embodiment of the present invention, the therapeutic agent comprises insulin and the TIC comprises antibodies specific for insulin. Data from the United States Renal Data System from 2000 indicated that 40% of dialysis patients are diabetic. Although not bound by theory, in certain individuals that have been subject to exogenous administration of insulin, antibodies have been generated that are specific for insulin. These antibodies bind insulin and form insoluble immune complexes with the exogenously administered insulin therapeutic. Thus, each injection or administration of insulin produces more of the insoluble immune complexes which then interfere with normal kidney operation (nephritis) and often result in kidney failure. Accordingly, pursuant to the present embodiment, a random selection of diabetic patients are tested on a periodic basis (monthly or longer) to determine the presence and/or levels of circulating anti-insulin antibodies. Based on the determination of the presence of anti-insulin antibodies in the subject therapy will be altered. Alternatively the circulating levels of anti-insulin antibodies will provide the information necessary to alter treatment. In this aspect, the subject will be assessed for an increased anti-insulin antibody titer as a result of the exogenous administration of insulin to the subject. Altered treatment for diabetic patients receiving exogenous insulin administration and that have developed anti-insulin antibodies can comprise administration of different forms of recombinant insulin or a pancreatic islet transplant. Other therapies exist including, for example, altering the diet of the patient to reduce insulin and the use of particulate inline blood filters are also contemplated to remove immune complex particulates. The present embodiment is useful as a guide for therapy for diabetic patients rather than a test for autoimmunity per se. These methods are useful to test diabetic patients, and monitoring them over time as they begin insulin treatment, moreover these methods are useful to test for the effectiveness of insulin administration and to prevent renal failure.

As another example embodiment, anemia may develop due to a decreased level or absence of erythropoietin. Erythropoietin is responsible for stimulating the proliferation and differentiation of erythroid progenitor cells in the bone marrow. A decrease or absence of erythropoietin production and/or activity may occur due to a variety of medical conditions, including chronic renal failure and in certain types of cancer. Such medical conditions give rise to the need for exogenous erythropoietin administration, such as recombinant human erythropoietin. Recombinant human erythropoietin (rhEPO) (e.g., darbepoetin alfa, EPOGEN® (epoetin alfa, Amgen, Inc., Thousand Oaks, Calif.), PROCRIT® (epoetin alfa, Ortho Biotech Products, L.P., Raritan, N.J.), NEORECORMON® (epoetin beta, Roche), EPREX® (epoetin alfa, Ortho Biologics, LLC), etc.), is widely used to support red blood cell production in subjects, including humans, cats, horses, dogs, etc. Therapeutic use of rhEPO in cats and dogs with erythropoietin-dependent anemia initially results in significant red blood cell response. However, the red blood cell status frequently declines after about 1 to 4 months despite continued rhEPO administration. Therapeutic failure of rhEPO in cats and dogs, estimated in clinical practice with an incidence between 20 and 50%, may result from interspecies variation in erythropoietin structure and the production of antibodies. Antibodies are thought not only to effectively block the bioactivity of recombinant EPO's, but also to cross-neutralize residual endogenous erythropoietin which leads to a pure red cell aplasia. Immunogenicity can be life threatening and has severely limited the therapeutic potential of rhEPO for various therapeutic applications. Furthermore, the development of antibodies to recombinant EPO can result in several global health-related problems in a subject. In no particular order, such antibodies may result in the simple ineffectiveness of the recombinant EPO drug. Immune precipitates from the combination of the antibodies with the recombinant EPO can also result, thus increasing the subject's chances for stroke, pulmonary embolism, clotting of blood resulting in an infarction, and/or destruction of any residual kidney function. Moreover, as mentioned, anaphylaxis and/or autoimmunity against patients own erythropoietin can occur, resulting in aplastic anemia In extreme circumstances, the person can be made dependant on erythropoietin to the extent where antibodies develop that destroy any residual EPO, and eventually all EPO exogenously administered, resulting in a life threatening circumstance. Thus, monitoring the concentration of erythropoietin is generally clinically indicated in order to avoid the development of autoimmunity to endogenous EPO thus causing a life threatening condition.

### VI. EXAMPLES

### Example 1

In one embodiment, a method is provided for testing a subject for the presence of, or monitoring a subject for the development of, antibodies to EPO. For example: (1) iodinate recombinant EPO and dilute with standard PTH tracer diluting buffer (available from Scantibodies Laboratory, Inc., Santee, Calif.), which buffer contains animal sera to prevent non-specific binding and buffer to ˜100,000 cpm/100 uL; (2) thaw 20 individual patient and 20 individual normal plasma samples; (3) add 200 uL of each sample to a tube (in duplicate); (4) add 100 uL of iodinated EPO to each tube containing sample, Vortex; (5) incubate at 35° C.-37° C. in a water bath for 2 hours; (6) after incubation, add 600 uL goat anti-human IgG to each tube, Vortex; (7) incubate each tube at room temperature for 2 hours; (8) centrifuge each tube for 30 minutes, at 2000 RPM, and 2-8C; and 9) decant liquid from each tube and count pellet for 1 minute on gamma counter. Samples containing antibodies to EPO elicited a increased signal compared with normal samples. See Table 1 below. As this table indicates, a comparison of the means between the normal samples and test subject samples indicates a defined increase in cpm for the test samples versus the normal samples. As also provided in Table 1, all test samples were obtained from dialysis patients. All test subjects are assumed to be receiving or have received recombinant EPO administration; although not bound by theory, currently EPO therapy comprises standard therapy for virtually all dialysis patients. See, e.g., J W Eschbach, et al., N. Engl. J. Med. 316:73-78 (1987); Robert S. Hillman, Hematopoietic Agents—Growth Factors, Minerals, and Vitamins, in THE PHARMACOLOGICAL BASIS OF THERAPEUTICS 1312-15 (JG Hardman, et al. eds., 9th ed. 1996); J. W. Greer et al., Health Care Financing Review, 20(3) (Spring 1999) (“In 1997 nearly all in-center hemodialysis patients received EPO regularly . . . ”). One of skill in the art would appreciate that reagents such as wash solutions and labels, as well as methods and devices utilized to detect bound labelled reagent can vary without departing from the gist and scope of the present embodiment. Although the present method utilizes thawed samples, fresh or otherwise unfrozen samples can also be utilized in accordance with the present method.

TABLE 1

| | |
|---|---|
| Normal Samples | Dialysis Patient Samples |

| Lot # | cpm | Subject # | cpm |
|---|---|---|---|
| F316A-1 | 5580 | 1 | 19371 |
| F655C-2 | 6715 | 2 | 11559 |
| F655C-3 | 6837 | 3 | 8053 |
| F655C-4 | 8197 | 4 | 14540 |
| F655C-5 | 7160 | 5 | 25854 |
| F655C-6 | 7041 | 6 | 13218 |
| F655C-7 | 16310 | 7 | 15047 |
| F655C-8 | 6517 | 8 | 16163 |
| F655C-9 | 5938 | 9 | 22938 |
| F655C-10 | 8930 | 10 | 20116 |
| F655C-11 | 11277 | 11 | 15143 |
| F655C-12 | 5704 | 12 | 14889 |
| F655C-13 | 14173 | 13 | 7986 |
| F655C-14 | 10668 | 14 | 17096 |
| F655C-15 | 8183 | 15 | 20489 |
| F655C-16 | 12635 | 16 | 19969 |
| F655C-17 | 12540 | 17 | 18992 |
| F655C-18 | 6269 | 18 | 22996 |
| F655C-19 | 13319 | 19 | 28032 |
| F655C-20 | 6293 | 20 | 17872 |
| Mean | 9014.3 | | 17516.15 |
| Std Dev | 3275.789 | | 5285.128 |

### Example 2

In another embodiment, a method is provided for testing a subject for the presence of, or monitoring a subject for the development of, antibodies to EPO. For example, biotinylated EPO is added to streptavidin coated microtiter plates (available from Scantibodies Laboratory, Inc.) and allowed to incubate. Sample suspected of (or known as) containing anti-EPO antibodies is then introduced to the plate and allowed to incubate. The plate is then washed. Labelled anti-human IgG and/or labelled anti-human IgM (utilizing, e.g., Isoluminol as a label) is then added to the plate and allowed to incubate for 2 hours at room temperature. The plate is then washed to remove unbound labelled anti-human IgG and/or labelled anti-human IgM, and the wells in the plate are examined (utilizing, e.g., a luminescent reader when labels such as Isoluminol are utilized) for bound labelled anti-human IgG and/or labelled anti-human IgM. One of skill in the art would appreciate that reagents such as wash solutions and labels, as well as methods and devices utilized to detect bound labelled reagent can vary without departing from the gist and scope of the present embodiment.

Frequently, one sample from an individual subject will be tested in accordance with the present methods. Also frequently, multiple samples each from individual subjects, are evaluated simultaneously in accordance with the present methods. Often, a known normal sample will be tested in parallel with the one or more samples obtained from test subjects. Moreover, one of skill in the art would recognize that one or more minor alterations to or substitutions within the above method will not depart from the gist and scope of the present embodiment.

In one embodiment, the therapeutic agent is recombinant erythropoietin or erythropoietin analog and the therapeutic inactivating component is an antibody specific for the recombinant erythropoietin or erythropoietin analog.

### Example 3

In another example, human antibody that is capable of specifically binding an antigen is separated from a reaction mixture for evaluation. The human antibody is separated via the introduction of goat anti-human IgG to the reaction mixture to form a complex, centrifugation of the reaction mixture containing goat-anti-human IgG to produce a pellet and decanting or aspirating the supernatant. The pellet is then assessed for the presence of human antibody. It was surprisingly recognized that the removal of the total fraction of human antibody allows for greater amplification and increased sensitivity than prior assays.

Further, in this embodiment unhindered antigen is utilized to form the reaction mixture such that it allows for full presentation, avoiding steric hindrance factors normally producing interferences that hinder the sensitivity and specificity of assays. In addition, a low molecular weight label, e.g., a radioactive type label such as Iodine-125, or a chemiluminescent type label such as luminol, is bound to the unhindered antigen. As further described below, the label preferably has a low molecular weight versus the molecular weight of the antigen. In a particularly preferred aspect, Iodine-125 is utilized as a labeling means having a defined point of attachment, e.g., attaching to the amino acids tyrosine and/or histidine, to the antigen. Moreover, Iodine-125 has an atomic weight of 125 daltons. For example, in the case of EPO, a 165 amino acid glycoprotein having a molecular weight of approximately 30,400 daltons (see, e.g., Sawyer, S. T. et al. Hematol. Oncol. Clinics NA 8:895 (1994); Jacobs, K. J. et al. Nature 313:806 (1985); and Lin, F-K. et al. Proc. Natl. Acad. Sci. USA 82:7580 (1985)) comprises the antigen. The EPO molecule has 4 tyrosines and 3 histidines. See Erythropoietin [Homo sapiens], NCBI Accession No. NP_{—}000790, version NP_{—}000790.1 GI:4503589. Since EPO contains 4 tyrosines and 3 histidines, a maximum of about 7 molecules of Iodine-125 could attached to, and thereby label, the EPO molecule. Thus, if all histidines and tyrosines were labelled with Iodine-125, the total additional molecular weight on the labelled EPO molecule would be 875 daltons. The utilization of Iodine-125 as a label would add less than 3% to the molecular weight of the unlabelled EPO molecule (e.g., 875/30,400=<3%). In comparison, if a label such as horse radish peroxidase, which has a molecular weight of 40,000 daltons, is conjugated to the EPO molecule, this would involve attachment of a label having a molecular weight comprising approximately 130% the weight of EPO itself. Thus, it was surprisingly recognized that using Iodine-125 as a label for molecules such as EPO is to use a label that is 20 times less hindering (by mass) than a conventional HRP label. Accordingly, the use of labelled EPO utilizing Iodine-125 label would allow much better presentation as an antigen than the use of EPO labelled with another label such as horseradish peroxidase. In the later circumstance the EPO molecule would be significantly hindered, in terms of steric hindrance. In contrast, the Iodine-125 form would be presented to the human antibody significantly unhindered. In this embodiment the antigen remains unhindered for at least two reasons, appropriate label selection and the use of unbound antigen. An appropriate label selection will result in the total mass of all label (or labelled reagent) bound to the antigen preferably totaling less than 50% of the mass of the antigen itself. Thus, in accordance with the presently contemplated embodiments, if the label is capable of binding the antigen in more than one location and enough label is bound to all positions on the antigen capable of being bound by that label, this total amount of bound label preferably totals less than about 50% of the mass of the antigen itself. The utilization of unbound antigen indicates that the antigen is not affixed to a solid phase (either from the start of the assay or mid-assay). Other labels and antigens, in addition to Iodine-125 and EPO, are further contemplated as described elsewhere herein. Moreover, in an occasional embodiment, a portion of the human antibody is separated from the reaction mixture and analyzed for a particular antigen.

The reaction mixture is formed via the introduction of a labeled unhindered antigen to a patient sample and allowing the human antibody, if present, to bind the labeled unhindered antigen. Additionally, goat anti-human IgG is introduced to the mixture of human antibody and labeled unhindered antigen after the human antibody is allowed to bind the labeled unhindered antigen to form a labeled complex. The human antibody comprised in the complex is then separated from the reaction mixture via precipitation. The complex is precipitated from the reaction mixture via centrifugation and aspirating or decanting the supernatant. Optionally, the reaction mixture is diluted utilizing a buffer prior to centrifugation. Also optionally, a detergent (e.g., Tween-20) is added to the reaction mixture prior to centrifugation. The precipitated labeled complex is then assessed for the presence and/or concentration of label. In the case of the use of Iodine-125 as a label, the precipitated labeled complex is assessed utilizing a gamma counter.

In a preferred aspect, the molecular weight of the label attached to the antigen totals less than 40%, less than 30%, less than 20%, or less than 10% of the molecular weight of the antigen itself (i.e., when not bound by label). In another preferred aspect, the molecular weight of the label attached to the antigen totals between about than 50% and 1% of the molecular weight of the antigen. In a particularly preferred aspect, the molecular weight of the label attached to the antigen totals between about 1% to about 3% of the molecular weight of the antigen. In another aspect, the molecular weight of the label attached to the antigen totals between about 1% to about 10% of the molecular weight of the antigen.

TABLE 2
Frequently Prescribed Drugs

| Brand Name | Manufacturer | Generic Name |
|---|---|---|
| ACCUPRIL ® | Parke-Davis | Quinapril |
| ACETAMINOPHEN/CODEINE | Various | Acetaminophen/Codeine |
| ACIPHEX ® | Eisai | Rabeprazole |
| ACTONEL ® | Procter & Gamble | Risedronate |
| ACTOS ® | Takeda | Pioglitazone |
| ACYCLOVIR | Various | Acyclovir |
| ADDERALL XR ® | Shire Rchwd | Amphetamine Mixed Salts |
| ADIPEX ® | Teva Pharm. | Phentermine |
| ADVAIR DISKUS ® | GlaxoSmithKline | Salmeterol/Fluticasone |
| ALBUTEROL AEROSOL | Various | Albuterol |
| ALBUTEROL SULFATE | Various | Albuterol |
| ALLEGRA ® | Hoech Mar R | Fexofenadine |
| ALLEGRA-D ® | Hoech Mar R | Fexofenadine/Pseudoephedrine |
| ALLOPURINOL | Various | Allopurinol |
| ALPRAZOLAM | Various | Alprazolam |
| ALTACE ® | Monarch | Ramipril |
| AMARYL ® | Hoech Mar R | Glimepiride |
| AMBIEN ® | Searle | Zolpidem |
| AMITRIPTYLINE | Various | Amitriptyline |
| AMOXICILLIN | Various | Amoxicillin |
| AMOXICILLIN/ | Various | Amoxicillin/Clavulanate |
| CLAVULANATE | | |
| AMOXIL ® | GlaxoSmithKline | Amoxicillin |
| APRI ® | Barr | Desogestrel/Ethinyl Estradiol |
| ASPIRIN | Various | Aspirin |
| ATENOLOL | Various | Atenolol |
| AUGMENTIN ES-600 ® | GlaxoSmithKline | Amoxicillin/Clavulanate |
| AUGMENTIN ® | GlaxoSmithKline | Amoxicillin/Clavulanate |
| AVANDIA ® | GlaxoSmithKline | Rosiglitazone maleate |
| AVAPRO ® | B-M Squibb | Irbesartan |
| AVIANE ® | Barr | Levonorgestrel/Ethinyl Estradiol |
| BACTROBAN ® | GlaxoSmithKline | Mupirocin |
| BEXTRA ® | Pharmacia/Upjohn | Valdecoxib |
| BIAXIN XL ® | Abbott | Clarithromycin |
| BIAXIN ® | Abbott | Clarithromycin |
| BISOPROLOL/HCTZ | Various | Bisoprolol/HCTZ |
| BUSPIRONE | Various | Buspirone |
| CAPTOPRIL | Various | Captopril |
| CARBIDOPA/LEVODOPA | Various | Carbidopa/Levodopa |
| CARISOPRODOL | Various | Carisoprodol |
| CARTIA XT ® | Andrx | Diltiazem |
| CEFZIL ® | B-M Squibb | Cefprozil |
| CELEBREX ® | Searle | Celecoxib |
| CELEBREX ® | Pharmacia | Celecoxib |
| CELEXA ® | Forest Pharm | Citalopram |
| CEPHALEXIN | Various | Cephalexin |
| CIPRO ® | Bayer Pharm | Ciprofloxacin |
| CLARINEX ® | Schering | Desloratadine |
| CLARITIN D 12 HR | Schering | Loratidine/Pseudoephedrine |
| CLARITIN D 24 HR ® | Schering | Loratidine/Pseudoephedrine |
| CLARITIN REDITABS ® | Schering | Loratadine |
| CLARITIN ® | Schering | Loratadine |
| CLINDAMYCIN | Various | Clindamycin |
| CLONAZEPAM | Various | Clonazepam |
| CLONIDINE | Various | Clonidine |
| COMBIVENT ® | Boehr lngel | Ipratropium/Albuterol |
| CONCERTA ® | Alza | Methylphenidate XR |
| COREG ® | GlaxoSmithKline | Carvedilol |
| COTRIM ® | Teva | Trimeth/Sulfameth |
| COUMADIN ® | Dupont | Warfarin |
| COZAAR ® | Merck | Losartan |
| CYCLOBENZAPRINE | Various | Cyclobenzaprine |
| DEPAKOTE ® | Abbott | Divalproex |
| DETROL LA ® | Pharmacia-Upjohn | Tolterodine |
| DIAZEPAM ® | Mylan | Diazepam |
| DICLOFENAC | Various | Diclofenac |
| DIFLUCAN ® | Pfizer | Fluconazole |
| DIGITEK ® | Bertek | Digoxin |
| DILANTIN ® | Parke-Davis | Phenytoin |
| DILTIAZEM HCL | Various | Diltiazem |
| DIOVAN HCT ® | Novartis | Valsartan/HCTZ |
| DIOVAN ® | Novartis | Valsartan |
| DITROPAN XL ® | Alza | Oxybutynin |
| DOXAZOSIN | Various | Doxazosin |
| DOXYCYCLINE HYCLATE | Various | Doxycycline |
| EFFEXOR XR ® | Wyeth-Ayerst | Venlafaxine |
| ENALAPRIL | Various | Enalapril |
| ENDOCET ® | Endo | Oxycodone/APAP |
| ERYPO ® | Ortho Biotech | Epoetin Alfa |
| ESTRADIOL | Various | Estradiol |
| EVISTA ® | Lilly | Raloxifene |
| FAMOTIDINE | Various | Famotidine |
| FIORICET ® | Sandoz | Butalbital |
| FLOMAX ® | Abbott | Tamsulosin |
| FLONASE ® | GlaxoSmithKline | Fluticasone |
| FLOVENT ® | GlaxoSmithKline | Fluticasone Propionate |
| FLUOXETINE | Various | Fluoxetine |
| FOLIC ACID | Various | Folic Acid |
| FORTEO ® | Eli Lilly | Teriparatide |
| FOSAMAX ® | Merck | Alendronate |
| FUROSEMIDE | Various | Furosemide |
| GEMFIBROZIL | Various | Gemfibrozil |
| GLIPIZIDE | Various | Glipizide |
| GLUCOPHAGE XR ® | B-M Squibb | Metformin |
| GLUCOPHAGE ® | B-M Squibb | Metformin |
| GLUCOTROL XL ® | Pfizer | Glipizide |
| GLUCOVANCE ® | B-M Squibb | Glyburide/Metformin |
| GLYBURIDE | Various | Glyburide |
| HUMALOG ® | Lilly | Insulin Lispro |
| HUMULIN 70/30 ® | Lilly | Human Insulin 70/30 |
| HUMULIN N ® | Lilly | Human Insulin NPH |
| HYDROCHLOROTHIAZIDE | Various | Hydrochlorothiazide |
| HYDROCODONE W/APAP | Various | Hydrocodone w/APAP |
| HYDROXYZINE HCL | Various | Hydroxyzine |
| HYZAAR ® | Merck | Losartan/HCTZ |
| IBUPROFEN | Various | Ibuprofen |
| IMITREX ORAL ® | GlaxoSmithKline | Sumatriptan |
| ISOSORBIDE MONONITRATE | Various | Isosorbide Mononitrate S.A. |
| KLOR-CON M20 ® | Upsher-Smith | Potassium Chloride |
| KLOR-CON ® | Upsher-Smith | Potassium Chloride |
| LANOXIN ® | GlaxoSmithKline | Digoxin |
| LASIX ® | Aventis | Furosemide |
| LEVAQUIN ® | McNeil | Levofloxacin |
| LEVOTHROID ® | Forest | Levothyroxine |
| LEVOXYL ® | Jones Medical Ind | Levothyroxine |
| LIPITOR ® | Pfizer | Atorvastatin Calcium |
| LISINOPRIL | Various | Lisinopril |
| LORAZEPAM | Various | Lorazepam |
| LOSEC ® | AstraZeneca | Omeprazole |
| LOTENSIN ® | Novartis | Benazepril |
| LOTREL ® | Novartis | Amlodipine/Benazepril |
| MACROBID ® | Procter & Gamble | Nitrofurantoin |
| MECLIZINE | Various | Meclizine |
| MEDROXYPROGESTERONE | Various | Medroxyprogesterone |
| METFORMIN | Various | Metformin |
| METHYLPREDNISOLONE | Various | Methylprednisolone |
| METOCLOPRAMIDE | Various | Metoclopramide |
| METOPROLOL TARTRATE | Various | Metoprolol |
| METRONIDAZOLE | Various | Metronidazole |
| MICROGESTIN FE ® | Watson | Norethindrone/Ethinyl Estradiol |
| MINOCYCLINE | Various | Minocycline |
| MIRCETTE ® | Organon | Desogestrel/Ethinyl Estradiol |
| MONOPRIL ® | B-M Squibb | Fosinopril |
| NAPROXEN | Various | Naproxen |
| NASACORT AQ ® | Hoech Mar R | Triamcinolone Acetonide |
| NASONEX ® | Schering | Mometasone |
| NECON ® | Watson | Ethinyl Estradiol/Norethindrone |
| NEURONTIN ® | Parke-Davis | Gabapentin |
| NEXIUM ® | AstraZeneca | Esomeprazole |
| NIFEDIPINE | Various | Nifedipine |
| NORTRIPTYLINE | Various | Nortriptyline |
| NORVASC ® | Pfizer | Amlodipine |
| NORVASC ® | Pfizer | Amlodipine |
| NYSTATIN | Various | Nystatin |
| OGASTRO ® | Tap Pharm | Lansoprazole |
| ORTHO TRI-CYCLEN ® | Ortho-McNeil | Norgestimate/Ethinyl Estradiol |
| ORTHO-CYCLEN ® | Ortho-McNeil | Norgestimate/Ethinyl Estradiol |
| ORTHO-NOVUM ® | Ortho-McNeil | Norethindrone/Ethinyl Estradiol |
| OXYCODONE/APAP | Various | Oxycodone/APAP |
| OXYCONTIN ® | Purdue | Oxycodone |
| PAXIL ® | GlaxoSmithKline | Paroxetine |
| PENICILLIN VK | Various | Penicillin VK |
| PHENTERMINE | various | Phentermine |
| PLAVIX ® | Sanofi | Clopidogrel |
| POTASSIUM CHLORIDE | Various | Potassium Chloride |
| PRAVACHOL ® | B-M Squibb | Pravastatin |
| PREDNISONE | Various | Prednisone |
| PREMARIN ® | Wyeth-Ayerst | Conjugated Estrogens |
| PREMPRO ® | Wyeth-Ayerst | Conj. Estrogens/Medroxyprogesterone |
| PREVACID ® | Tap Pharm | Lansoprazole |
| PRILOSEC ® | AstraZeneca | Omeprazole |
| PRINIVIL ® | Merck | Lisinopril |
| PROMETHAZINE | Various | Promethazine |
| PROMETHAZINE/CODEINE | Various | Promethazine/Codeine |
| PROPACET | Various | Propoxyphene N/APAP |
| PROPOXYPHENE N/APAP | Various | Propoxyphene N/APAP |
| PROPRANOLOL | Various | Propranolol |
| PROSTASCINT ® | Cytogen Corp | Capromab Pendetide |
| PROTONIX ® | Wyeth-Ayerst | Pantoprazole |
| RANITIDINE HCL | Various | Ranitidine |
| REMERON ® | Organon | Mirtazapine |
| RHINOCORT AQUA ® | AstraZeneca | Budesonide |
| RISPERDAL ® | Janssen | Risperidone |
| | Pharmaceutica | |
| | Products, LP | |
| SEREVENT ® | GlaxoSmithKline | Salmeterol |
| SEROQUEL ® | AstraZeneca | Quetiapine |
| SEROXAT ® | GlaxoSmithKline | Paroxetine |
| SINGULAIR ® | Schein | Montelukast |
| SKELAXIN ® | Elan | Metaxalone |
| SOMA ® | various | Carisoprodol |
| SPIRONOLACTONE | Various | Spironolactone |
| SYNTHROID ® | Knoll | Levothyroxine |
| TAMOXIFEN | Various | Tamoxifen |
| TEMAZEPAM | Various | Temazepam |
| TERAZOSIN | Various | Terazosin |
| TETRACYCLINE | Various | Tetracycline |
| TIAZAC ® | Forest | Diltiazem |
| TIMOLOL MALEATE | Various | Timolol Maleate |
| TOPAMAX ® | Ortho-McNeil | Topiramate |
| TOPROL-XL ® | AstraZeneca | Metoprolol |
| TRAMADOL | Various | Tramadol |
| TRAZODONE | Various | Trazodone |
| TRIAMCINOLONE | Various | Triamcinolone |
| ACETONIDE | | |
| TRIAMTERENE/HCTZ | Various | Triamterene/HCTZ |
| TRICOR ® | Abbott | Fenofibrate |
| TRIMETHOPRIM/ | Various | Trimeth/Sulfameth |
| SULFAMETHOXAZOLE | | |
| TRIMOX ® | Apothecon | Amoxicillin |
| TRIVORA-28 ® | Watson | Levonorgestrel/Ethinyl Estradiol |
| ULTRACET ® | Ortho-McNeil | Tramadol/Acetaminophen |
| ULTRAM ® | McNeil | Tramadol |
| ULTRAM ® | Johnson & Johnson | Tramadol Hydrochloride |
| VALTREX ® | GlaxoSmithKline | Valacyclovir |
| VEETIDS ® | Apothecon | Penicillin VK |
| VERAPAMIL HCL | Various | Verapamil |
| VIAGRA ® | Pfizer | Sildenafil Citrate |
| VIOXX ® | Merck & Co. | Rofecoxib |
| WARFARIN | Various | Warfarin |
| WELLBUTRIN SR ® | Glaxo Well | Bupropion HCL |
| XALATAN ® | Pharmacia/Upjohn | Latanoprost |
| ZEMPLAR ® | Abbott | Paricalcitol |
| ZESTORETIC ® | AstraZeneca | Lisinopril/HCTZ |
| ZESTRIL ® | AstraZeneca | Lisinopril |
| ZITHROMAX ® | Pfizer | Azithromycin |
| ZOCOR ® | Merck | Simvastatin |
| ZOLOFT ® | Pfizer | Sertraline |
| ZYPREXA ® | Lilly | Olanzapine |
| ZYPREXA ® | Eli Lilly | Olanzapine |
| ZYRTEC ® | Pfizer | Cetirizine |

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method of monitoring the therapeutic inactivating capacity of a subject, said method comprising:
a) obtaining a sample from a subject to be monitored;
b) assessing said sample for a therapeutic inactivating component specific for a therapeutic agent that has been administered, is being administered or will be administered to said subject, wherein said therapeutic inactivating component binds with said therapeutic agent and interferes with the utility of said therapeutic agent, with aproviso that when said therapeutic inactivating component is an antibody, said antibody is not assessed via plasmon resonance; and
c) deciding to initiate, terminate, or adjust the level of administration of said therapeutic agent to said subject based on said assessed therapeutic inactivating component.

2. The method of claim 1, wherein the subject is afflicted by or believed to be afflicted by a medical condition.

3. The method of claim 2, wherein the therapeutic agent is useful for treating the medical condition or the underlying symptomology of the medical condition.

4. The method of claim 1, wherein the therapeutic agent is selected from the group provided in Table 2.

5. The method of claim 3, wherein the therapeutic agent is selected from the group consisting of a pain management agent, an antipyretic agent, a migraine agent, a prophylaxis agent, an anti-infective agent, an (anti-)inflammatory agent, an (anti-)parasitic agent, a uterine agent, a (anti-) microbial agent, an (anti-)arthritic agent, a gout related agent, a cardiovascular agent, a cancer agent, an immunomodulation agent, a metabolic agent, a musculoskeletal agent, a (anti-)toxicity agent, a dermatologic agent, an ophthalmic agent, an otic agent, a pharyngeal agent, a nasal agent, an HIV or AIDS related agent, an allergy or asthma related agent, an Alzheimer's disease related agent, a diabetes related agent, a glandular disorder related agent, a kidney disease related agent, a liver disease related agent, a mental health related agent, an osteoporosis related agent, a Parkinson's disease related agent, an osteoporosis related agent, a renal bone disease agent, an agent to treat disorders of the parathyroid gland, a sexually transmitted disease related agent, a stroke related agent, a blood or circulatory related agent, an endocrine related agent, a gastrointestinal agent, a neurological agent, and a respiratory agent.

6. The method of claim 3, wherein the medical condition is selected from the group consisting of a pain management related condition, a migraine related condition, an infection, an inflammatory related condition, a urinary related condition, an OB/GYN disorder, an arthritic related condition, a foot related condition, a cardiovascular related condition, a metabolic related condition, a musculoskeletal related condition, cancer, an immunological related condition, a toxicity related condition, a dermatologic related condition, an ophthalmic related condition, an otic related condition, a pharyngeal related condition, a nasal related condition, a blood or circulatory related condition, AIDS, allergy & asthma, Alzheimer's disease, a child specific condition, diabetes, a glandular disorder, kidney disease, liver disease, mental health related condition, osteoporosis, renal bone disease, Parkinson's disease, a sexually transmitted disease, stroke, an endocrine related condition, a gastrointestinal related condition, a neurological related condition, and a respiratory related condition.

7. The method of claim 1, wherein multiple therapeutic agents are identified and the biological sample is assessed for a therapeutic inactivating component specific for a selection of the therapeutic agent(s).

8. The method of claim 1, wherein the interference with the utility of said therapeutic agent comprises reduced or eliminated efficacy, or an adverse biological reaction to the therapeutic agent.

9. The method of claim 8, wherein the adverse biological reaction comprises the generation of an immune response to the therapeutic agent.

10. The method of claim 1, wherein the presence or absence of the therapeutic inactivating component is assessed.

11. The method of claim 1, wherein the level of the therapeutic inactivating component is assessed.

12. The method of claim 1, wherein the therapeutic inactivating component is assessed by a sandwich or competitive assay format.

13. The method of claim 1, wherein the therapeutic inactivating component is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), electron transfer assay, complement fixation, indirect immunofluorescent assay (IFA), nephelometry, flow cytometry assay, chemiluminescence assay, lateral flow immunoassay, u-capture assay, inhibition assay and avidity assay.

14. The method of claim 1, wherein the therapeutic agent is a small molecule or biomolecule.

15. The method of claim 1, wherein the therapeutic agent is recombinant erythropoietin or an erythropoietin analog.

16. The method of claim 1, wherein the therapeutic inactivating component is an antibody or antibody fragment.

17. The method of claim 2, wherein the therapeutic agent is available for treating the medical condition, and the decision to initiate, terminate, or adjust the level of any one of the one or more therapeutic agents is based on said assessed therapeutic inactivating component.

18. The method of claim 1, wherein the decision for initiating, terminating or adjusting the level of administration of the therapeutic agent to the subject is made by a health care provider or a personnel of a health care management entity.

19. The method of claim 18, wherein the health care provider is a clinician or a nurse.

20. The method of claim 1, which is conducted in a clinical lab.

21. The method of claim 1, wherein the therapeutic agent is selected from the group consisting of atorvastatin, epoetin alfa, paricalcitol, risperidone, a calcimemetic, furosemide, a bisphosphanate, and teriparatide.

22. The method of claim 1, wherein the subject has previously received and is currently receiving administration of the therapeutic agent, wherein the therapeutic inactivating component is an antibody, and wherein the sample is assessed for the presence or absence of the antibody.

23. The method of claim 22, wherein the subject is monitored over a prolonged period of treatment with the therapeutic agent for the development of the antibody.

24. The method of claim 23, wherein the therapeutic agent is recombinant erythropoietin or an erythropoietin analog.

25. The method of claim 1, wherein the sample is assessed via steps comprising:
a) contacting the sample containing the therapeutic inactivating component, if any, with the therapeutic agent to form a reaction mixture, wherein the therapeutic agent comprises an unhindered form of the therapeutic agent bound by a low molecular weight label, and wherein the therapeutic inactivating component, if any, binds the unhindered, labeled therapeutic agent to form a labeled therapeutic inactivating component complex;
b) separating the labeled therapeutic inactivating component complex from the reaction mixture; and
c) assessing the separated labeled therapeutic inactivating component complex.

26. The method of claim 25, wherein the proportion of the molecular weight of the low molecular weight label bound to the unhindered therapeutic agent, versus the molecular weight of the unhindered therapeutic agent itself, comprises less than about 50%.

27. A method for determining or monitoring a therapeutic protocol for a subject afflicted with an auto antibody specific for a natural substance, wherein said auto antibody developed as a result of therapeutic administration of the natural substance or an analog thereof, said method comprising:
a) obtaining a sample from said subject;
b) assessing said sample for the presence and/or level of said natural substance;
c) assessing said sample for the presence of said auto antibody that specifically binds said natural substance, wherein said auto antibody is not assessed via plasmon resonance or a competitive assay, and said natural substance is not insulin or thyroglobulin; and
d) deciding to initiate, terminate, or adjust the level of administration of the natural substance to said subject based on said assessed auto antibody.

28. The method of claim 27, wherein the natural substance is useful for treating the medical condition or the underlying symptomology of the medical condition.

29. The method of claim 27, wherein the natural substance is selected from the group provided in Table 2.

30. The method of claim 27, wherein the presence or absence of the auto antibody is assessed.

31. The method of claim 27, wherein the auto antibody is assessed by a sandwich assay format.

32. The method of claim 27, wherein the decision for initiating, terminating or adjusting the level of administration of the therapeutic agent to the subject is made by a health care provider or a personnel of a health care management entity.

33. The method of claim 27, which is conducted in a clinical lab.

34. The method of claim 27, wherein the natural substance is parathyroid hormone (PTH).

35. The method of claim 27, wherein the therapeutic agent comprises a compound having an antagonistic biological effect to that normally exhibited by the natural substance.

36. The method of claim 27, wherein the auto antibody is specific for a receptor involved in a biological pathway affected by the natural substance.

37. The method of claim 36, wherein the receptor is a calcium sensing receptor and the natural substance is PTH.

38. The method of claim 27, wherein the natural substance is erythropoietin.

39. The method of claim 27, wherein the sample is assessed for the presence of the auto antibody via steps comprising:
a) contacting the sample containing the auto antibody, if any, with the natural substance to form a reaction mixture, wherein the natural substance comprises an unhindered form of the natural substance bound by a low molecular weight label, and wherein the auto antibody, if any, binds the unhindered, labeled natural substance to form a labeled auto antibody complex;
b) separating the labeled auto antibody complex from the reaction mixture; and
c) assessing the separated labeled auto antibody complex.

40. The method of claim 39, wherein the proportion of the molecular weight of the low molecular weight label that is capable of binding the unhindered natural substance, versus the molecular weight of the unhindered natural substance itself, comprises less than about 50%.

41. A method for determining or monitoring a therapeutic protocol for a subject receiving or about to receive administration of a chemical moiety-based therapeutic agent, said method comprising:
a) obtaining a sample from said subject;
b) assessing said sample for a therapeutic inactivating component specific for said chemical moiety-based therapeutic agent that has been administered, is being administered or will be administered to said subject, wherein said therapeutic inactivating component binds with said chemical moiety-based therapeutic agent and interferes with the utility of said therapeutic agent; and
c) deciding to initiate, terminate, or adjust the level of administration of said chemical moiety-based therapeutic agent to said subject based on said assessed therapeutic inactivating component.

42. The method of claim 41, wherein the chemical moiety-based therapeutic agent is a small molecule.

43. The method of claim 41, wherein the chemical moiety-based therapeutic agent is a prescription drug or an over the counter drug.

44. The method of claim 41, wherein the presence or absence of the therapeutic inactivating component is assessed.

45. The method of claim 41, wherein the decision for initiating, terminating or adjusting the level of administration of the chemical moiety-based therapeutic agent to the subject is made by a health care provider or a personnel of a health care management entity.

46. The method of claim 41, which is conducted in a clinical lab.

47. The method of claim 41, wherein the subject has previously received and is currently receiving administration of the chemical moiety-based therapeutic agent, wherein the therapeutic inactivating component is an antibody, and wherein the sample is assessed for the presence or absence of the antibody.

48. The method of claim 47, wherein the subject is monitored over a prolonged period of treatment with the chemical moiety-based therapeutic agent for the development of the antibody.

49. The method of claim 41, wherein the sample is assessed via steps comprising:
a) contacting the sample containing the therapeutic inactivating component, if any, with the chemical moiety-based therapeutic agent to form a reaction mixture, wherein the chemical moiety-based therapeutic agent comprises an unhindered form of the chemical moiety-based therapeutic agent bound by a low molecular weight label, and wherein the therapeutic inactivating component, if any, binds the unhindered, labeled chemical moiety-based therapeutic agent to form a labeled therapeutic inactivating component complex;
b) separating the labeled therapeutic inactivating component complex from the reaction mixture; and
c) assessing the separated labeled therapeutic inactivating component complex.

50. The method of claim 49, wherein the proportion of the molecular weight of the low molecular weight label that is capable of binding the unhindered chemical moiety-based therapeutic agent, versus the molecular weight of the unhindered chemical moiety-based therapeutic agent itself, comprises less than about 50%.

51. A kit for monitoring therapeutic inactivating capacity of a subject, which kit comprises:
a) a means for assessing therapeutic inactivating component of a sample obtained from a subject to a therapeutic agent that has been administered, is being administered or will be administered to said subject; and
b) instructions for making a decision for initiating, terminating or adjusting the level of administration of said therapeutic agent to said subject based on said assessed therapeutic inactivating component.

52. The kit of claim 51, which further comprises a means for obtaining a sample from a subject to be monitored.

53. The kit of claim 51, wherein the therapeutic agent is selected from those listed in Table 2.

54. The kit of claim 51, wherein the means for assessing a therapeutic inactivating component comprises an unhindered therapeutic agent, and a low molecular weight label bound to the unhindered therapeutic agent.

55. The kit of claim 51, wherein the proportion of the molecular weight of the low molecular weight label that is capable of binding the unhindered therapeutic agent, versus the molecular weight of the unhindered therapeutic agent itself, comprises less than about 50%.

56. A kit comprising:
a) a therapeutic agent; and
b) instructions for monitoring the therapeutic inactivating capacity of a subject to said therapeutic agent.

57. The kit of claim 56, wherein the therapeutic agent is an unhindered therapeutic agent, and the kit further comprises a low molecular weight label bound to the unhindered therapeutic agent.

58. A method of monitoring the hormone inactivating capacity of a subject, said method comprising:
a) obtaining a sample from a subject to be monitored;
b) assessing said sample for a hormone inactivating component specific for a hormone, wherein said hormone inactivating component binds with said hormone and interferes with the normal biological activity of said hormone; with aproviso that when said hormone inactivating component is an antibody, said antibody is not assessed via plasmon resonance, and a further proviso that when said hormone is insulin or thyroglobulin, said hormone inactivating component is not an auto antibody; and
c) deciding to initiate, terminate, or adjust the level of therapeutic administration of said hormone to said subject based on said assessed hormone inactivating component.

59. The method of claim 58, wherein the hormone is recombinant erythropoietin or erythropoietin analog and said hormone inactivating component is an antibody specific for the recombinant erythropoietin or erythropoietin analog.

60. The method of claim 58, wherein the subject has previously received and is currently receiving administration of the hormone, wherein the therapeutic inactivating component is an antibody, and wherein the sample is assessed for the presence or absence of the antibody.

61. The method of claim 60, wherein the subject is monitored over a prolonged period of treatment with the hormone for the development of the antibody.

62. The method of claim 61, wherein the hormone is recombinant erythropoietin or an erythropoietin analog.

63. The method of claim 58, wherein the sample is assessed via steps comprising:
a) contacting the sample containing the hormone inactivating component, if any, with the hormone to form a reaction mixture, wherein the hormone comprises an unhindered form of the hormone bound by a low molecular weight label, and wherein the hormone inactivating component, if any, binds the unhindered, labeled hormone to form a labeled hormone inactivating component complex;
b) separating the labeled hormone inactivating component complex from the reaction mixture; and
c) assessing the separated labeled hormone inactivating component complex.

64. The method of claim 63, wherein the proportion of the molecular weight of the low molecular weight label that is capable of binding the unhindered hormone, versus the molecular weight of the unhindered hormone itself, comprises less than about 50%.
